(19) United States
(12) Patent Application Publication
(10) Pub. No.: US 2026/0315205 A1
(43) Pub. Date: Oct. 15, 2026

(54) Cannulation method

(71) Applicant: OLYMPUS MEDICAL SYSTEMS CORP., Tokyo (JP)

(72) Inventors: Takahira TOKIMOTO, Hachioji-shi (JP); Tomoyasu HASEGAWA, Hachioji-shi (JP); Chisato KIKUCHI, Tokyo (JP); Takuo YOKOTA, Hino-shi (JP); Ko TAKITA, Hachioji-shi (JP); Kunihide KAJI, Hachioji-shi (JP); Keita SHOJI, Hino-shi (JP); Shinichi KITADE, Hino-shi (JP); Kazuhiro KAN, Kunitachi-shi (JP)

(73) Assignee: OLYMPUS MEDICAL SYSTEMS CORP., Tokyo (JP)

(21) Appl. No.: 19/539,499

(22) Filed: Feb. 13, 2026

Related U.S. Application Data
(60) Provisional application No. 63/758,419, filed on Feb. 14, 2025.

(51) Int. Cl. A61F 2/04; A61F 2/958

(52) U.S. Cl. CPC A61F 2/04; A61F 2/958; A61F 2002/041

(57) ABSTRACT

A cannulation method includes inserting an endoscope close to the papilla, deforming the papilla or the bile pancreatic duct, and inserting an insertion device into the bile pancreatic duct. The papilla may be manipulated to move the opening portion of the bile pancreatic duct to a position visible through the endoscope, and the insertion device may be inserted into the opening portion.

### TECHNICAL FIELD

The present disclosurerelates to a cannulation method and an endoscopic treatment tool. Priority is claimed on US Provisional Patent Application No. 63/758,419, filed Feb. 14, 2025, the content of which is incorporated herein by reference.

### BACKGROUND

A procedure is performed to insert a medical device such as a guide wire into the bile duct. The bile duct has narrowed and tortuous sections, making it difficult to insert the medical device into the bile duct. As described in US Patent Publication No. 2025/0169842, a medical device has been devised that is easy to insert into the bile duct.

### SUMMARY

A cannulation method includes inserting an endoscope close to the papilla, deforming the papilla or bile pancreatic duct, and inserting an insertion device into the bile pancreatic duct.

### BRIEF DESCRIPTION OF THE DRAWINGS

FIG. 1 An overall view of an endoscope treatment system according to a first embodiment.

FIG. 2 A view showing a bile duct B of a treatment target.

FIG. 3 A view showing an example of a biomanipulation process.

FIG. 4 A view showing an example of the biomanipulation process.

FIG. 5 A view showing an example of the biomanipulation process.

FIG. 6 A view showing an example of the biomanipulation process.

FIG. 7 A view showing an example of the biomanipulation process.

FIG. 8 A view showing an example of the biomanipulation process.

FIG. 9 A view showing an example of the biomanipulation process.

FIG. 10 A view showing an example of the biomanipulation process.

FIG. 11 A view showing an example of the biomanipulation process.

FIG. 12 A view showing an example of the biomanipulation process.

FIG. 13 A view showing an example of the biomanipulation process.

FIG. 14 A view showing an example of the biomanipulation process.

FIG. 15 A view showing an example of the biomanipulation process.

FIG. 16 A view showing an example of the biomanipulation process.

FIG. 17 A view showing an example of the biomanipulation process.

FIG. 18 A view showing an example of the biomanipulation process.

FIG. 19 A view showing an example of the biomanipulation process.

FIG. 20 A view showing an example of the biomanipulation process.

FIG. 21 A view showing an example of the biomanipulation process.

FIG. 22 A view showing an example of the biomanipulation process.

FIG. 23 A view showing an example of the biomanipulation process.

FIG. 24 A view showing an example of the biomanipulation process.

FIG. 25 A view showing an example of the biomanipulation process.

FIG. 26 A view showing an example of the biomanipulation process.

FIG. 27 A view showing an example of the biomanipulation process.

FIG. 28 A view showing an example of the biomanipulation process.

FIG. 29 A view showing an example of the biomanipulation process.

FIG. 30 A view showing an example of a bile duct intubation process.

FIG. 31 A view showing an example of the bile duct intubation process.

FIG. 32 A view showing an example of the bile duct intubation process.

FIG. 33 A view showing an example of the bile duct intubation process.

FIG. 34 A view showing an example of the bile duct intubation process.

FIG. 35 A view showing an example of the bile duct intubation process.

FIG. 36 A view showing an example of the bile duct intubation process.

FIG. 37 A view showing an example of the bile duct intubation process.

FIG. 38 A view showing an example of the bile duct intubation process.

FIG. 39 A view showing an example of the bile duct intubation process.

FIG. 40 A view showing an example of the bile duct intubation process.

FIG. 41 A view showing an example of the bile duct intubation process.

FIG. 42 A view showing an example of the bile duct intubation process.

FIG. 43 A view showing an example of the bile duct intubation process.

FIG. 44A A view showing an example of the bile duct intubation process.

FIG. 44B A view showing an example of the bile duct intubation process.

FIG. 45 A view showing an example of the bile duct intubation process.

FIG. 46 A view showing a variant of a tip portion.

FIG. 47 A view showing an example of the bile duct intubation process.

FIG. 48 A view showing an example of the bile duct intubation process.

FIG. 49 A view showing an example of the bile duct intubation process.

FIG. 50 A view showing an example of the biomanipulation process.

FIG. 51 A view showing an example of the biomanipulation process.

FIG. 52 A view showing an example of the biomanipulation process.

FIG. 53 A view showing an example of the biomanipulation process.

FIG. 54 A view showing an example of the biomanipulation process.

FIG. 55 A view showing an example of the biomanipulation process.

FIG. 56 A view showing an example of the biomanipulation process.

FIG. 57 A view showing an example of the biomanipulation process.

FIG. 58 A view showing an example of the biomanipulation process.

FIG. 59 A view showing an example of the biomanipulation process.

FIG. 60 A view showing an example of the bile duct intubation process.

FIG. 61 A view showing an example of the bile duct intubation process.

FIG. 62 A view showing an example of the bile duct intubation process.

FIG. 63 A view showing an example of the bile duct intubation process.

FIG. 64 A view showing an example of the bile duct intubation process.

FIG. 65 A view showing an example of the bile duct intubation process.

FIG. 66 A view showing an example of the bile duct intubation process.

FIG. 67 A view showing an example of the bile duct intubation process.

FIG. 68 A view showing an example of the bile duct intubation process.

FIG. 69 A view showing an example of the bile duct intubation process.

FIG. 70 A view showing an example of the bile duct intubation process.

FIG. 71 A view showing an example of the bile duct intubation process.

FIG. 72 A view showing an example of the bile duct intubation process.

FIG. 73 A view showing an example of the bile duct intubation process.

FIG. 74 A view showing an example of the bile duct intubation process.

FIG. 75 A view showing an example of the bile duct intubation process.

FIG. 76 A view showing an example of the bile duct intubation process.

FIG. 77 A view showing an example of the bile duct intubation process.

FIG. 78 A view showing an example of the bile duct intubation process.

FIG. 79 A view showing an example of the bile duct intubation process.

FIG. 80 A view showing an example of the bile duct intubation process.

FIG. 81 A view showing an example of the bile duct intubation process.

FIG. 82 A view showing an example of the bile duct intubation process.

FIG. 83 A view showing an example of the bile duct intubation process.

FIG. 84 A view showing an example of the bile duct intubation process.

FIG. 85 A view showing an example of an observation process.

FIG. 86 A view showing an example of the observation process.

FIG. 87 A view showing an example of the observation process.

FIG. 88 A view showing an example of the observation process.

FIG. 89 A view showing an example of the observation process.

FIG. 90 A view showing an example of the observation process.

FIG. 91 A view showing an example of the observation process.

FIG. 92 A view showing an example of an insertion process.

FIG. 93 A view showing an example of the insertion process.

FIG. 94 A view showing an example of the insertion process.

FIG. 95 A view showing an example of the insertion process.

FIG. 96 A view showing an example of a pancreatic duct obstruction process.

FIG. 97 A view showing an example of the insertion process.

FIG. 98 A view showing an example of the insertion process.

FIG. 99 A view showing an example of the insertion process.

FIG. 100A A view showing an example of the insertion process.

FIG. 100B A view showing an example of the insertion process.

FIG. 100C A view showing an example of the insertion process.

FIG. 101 A view showing an example of the insertion process.

FIG. 102 A view showing an example of the insertion process.

FIG. 103 A view showing an example of the insertion process.

FIG. 104 A view showing an example of the insertion process.

FIG. 105 A view showing an example of the insertion process.

FIG. 106 A view showing an example of the insertion process.

### DETAILED DESCRIPTION

### First Embodiment

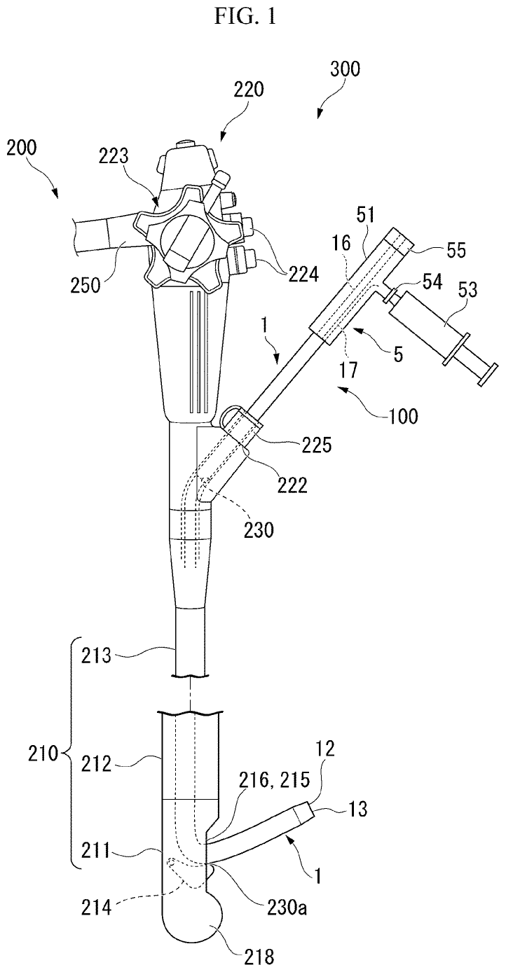

A cannulation method according to a first embodiment will be described with reference to FIG. 1 to FIG. 29.

### [Endoscope Treatment System 300]

An endoscope treatment system 300 includes a catheter 100, and an endoscope 200. The catheter 100 is inserted into the endoscope 200 and used.

### [Endoscope 200]

The endoscope 200 is a side-viewing flexible endoscope capable of optical observation and ultrasonic observation. The endoscope 200 includes an elongated insertion section 210, an operation part 220 provided on a base end of the insertion section 210, and a universal cord 250 extending from the operation part 220. Further, the endoscope 200 may be a direct-viewing flexible endoscope.

The insertion section 210 has a tip portion 211 provided on a tip thereof, a curved section 212 provided on a base end side of the tip portion 211 and enabling a bending operation, and a flexible tube section 213 provided on a base end side of the curved section 212. The insertion section 210 is formed with a treatment tool channel 230 into which an endoscopic treatment tool such as the catheter 100 or the like can be inserted.

A side surface of the tip portion 211 is provided with a light guide 215, an optical observation part 216 having an image sensor such as a CCD or the like, and a tip opening 230a in communication with the treatment tool channel 230.

A standing platform 214 is provided in the vicinity of the tip opening 230a of the tip portion 211. A base end portion of the standing platform 214 is rotatably supported by the tip portion 211. A standing platform operating wire (not shown) fixed to a tip portion of the standing platform 214 extends to the operation part 220 through the inside of the insertion section 210.

An ultrasonic transducer 218 is provided on a tip of the tip portion 211. Further, the ultrasonic transducer 218 may be provided closer to the base end than the tip opening 230a.

The curved section 212 is freely curved in an upward/downward direction and a leftward/rightward direction. A tip of an operating wire is fixed to a tip side of the curved section 212. The operating wire extends to the operation part 220 through the inside of the insertion section 210.

The operation part 220 is provided with a knob 223 configured to operate the operating wire or the standing platform operating wire, and a switch 224 configured to operate the optical observation part 216 or the like. A surgeon can bend the curved section 212 in a desired direction by operating the knob 223.

The operation part 220 is provided with a forceps port (base end opening) 222 in communication with the treatment tool channel 230. The surgeon can insert an endoscopic treatment tool such as the catheter 100 or the like from the forceps port 222. A forceps plug 225 configured to prevent a leakage of a body fluid is attached to the forceps port 222.

The universal cord 250 connects the endoscope 200 to a video control device. An imaging signal captured by the optical observation part 216 is transmitted to the video control device via the universal cord 250.

### [Catheter 100]

The catheter 100 includes a sheath 1, and an operation part 5. In the following description, a side of the catheter 100 inserted into a patient's lumen in a longitudinal direction A is referred to as “a tip side (distal side) A1,” and a side of the operation part 5 is referred to as “a base end side (proximal side) A2.”

The sheath 1 is an elongated tubular member that can be inserted through the treatment tool channel 230 of the endoscope 200. The sheath 1 is formed with a guide wire lumen 16 through which a guide wire GW is inserted, and a contrast lumen 17 through which a contrast agent is inserted.

The tip portion 11 is a hard tube-shaped member provided at the tip of the sheath 1. The tip portion 11 may be a metal member or a plastic member reinforced with glass or carbon. A tip 1d of the tip portion 11 is formed with a tip opening 12 in communication with the guide wire lumen 16, and a tip opening 13 in communication with the contrast lumen 17.

The operation part 5 has an operation part main body 51 connected to a base end of the sheath 1, and a syringe 53 connected to the operation part main body 51.

The operation part main body 51 is formed in a tubular shape. The operation part main body 51 has an opening 54 to which the syringe 53 is connected, and a guide wire port 55 through which the guide wire GW is inserted.

The opening 54 is an opening to which the syringe 53 is attached and communicates with the contrast lumen 17. Fluids such as degassed water, physiological saline, and contrast agent supplied from the syringe 53 flow through the contrast lumen 17 of the sheath 1 and out of the tip opening 13 of the tip portion 11.

The guide wire port 55 is an opening through which the guide wire GW is inserted and is in communication with the guide wire lumen 16. The guide wire GW inserted from the guide wire port 55 passes through the guide wire lumen 16 and protrudes from the tip opening 12 of the tip portion 11.

### [Operations of Endoscope Treatment System 300]

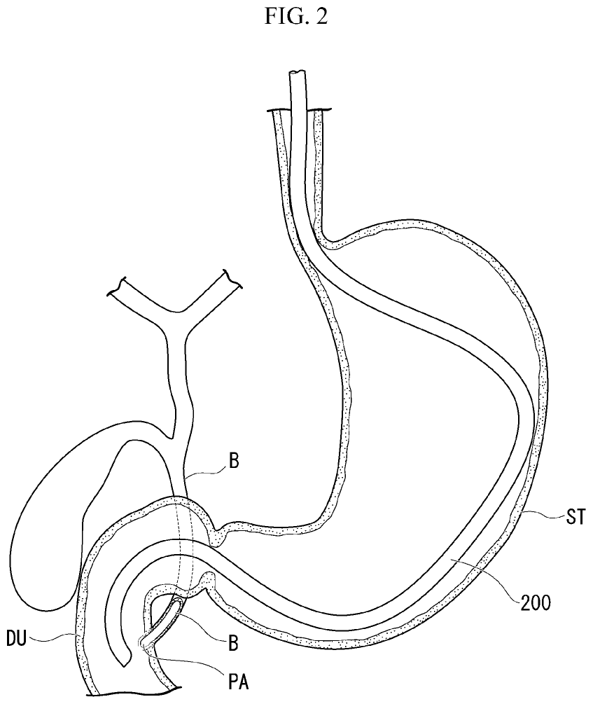

Next, actions and effects of the endoscope treatment system 300 according to the embodiment will be described with reference to FIG. 2 to FIG. 29. Specifically, a procedure for treating the bile duct B using endoscopic retrograde cholangiopancreatography (ERCP) will be described. FIG. 2 is a view showing the bile duct B of the treatment target.

### <Step S1: Endoscope Insertion Process>

In step S1, the surgeon inserts the insertion section 210 of the endoscope 200 into the patient's lumen through a natural opening such as the mouth. The surgeon operates the operation part 220 as needed to curve the curved section 212. The surgeon inserts the tip portion 211 of the endoscope 200 to the vicinity of the duodenal papilla.

### <Step S2: Endoscope Positioning Process>

In step S2, the surgeon adjusts the position of the tip portion 211 of the endoscope 200 so that the duodenal papilla is within an imaging range of the optical observation part 216 of the endoscope 200. The surgeon adjusts the position of the tip portion 211 by appropriately combining forward/backward operations, bending operations, twisting operations, etc. of the endoscope 200.

### <Step S3: Biomanipulation Process>

In the case of the papilla PA, such as an intradiverticular papilla, it may be difficult to direct the catheter 100 into an opening portion PA1 of the papilla PA. In such a case, the surgeon manipulates the papilla PA to move the opening portion PA1 of the papilla PA to a position visible from the endoscope 200. After the opening portion PA1 can be observed, the catheter 100 is inserted into the opening portion PA1.

FIG. 3 is a view showing an example of a biomanipulation process.

For example, as shown in FIG. 3, the surgeon pulls a tube wall with a threaded clip device 310 to move the opening portion PA1 to a position visible from the endoscope 200. Specifically, the surgeon inserts the threaded clip device 310 into the treatment tool channel 230 so that it protrudes from the tip opening 230a of the treatment tool channel 230. Next, the surgeon grasps the tube wall in the diverticulum with the threaded clip device 310 and pulls it out, moving the papilla PA to a position visible from inside the duodenum. Next, the surgeon pulls the thread of the threaded clip device 310, presses the thread against the duodenum wall, and then grips the tube wall along with the thread with the clip, fixing the papilla PA facing the inside of the duodenum. Next, the surgeon removes the threaded clip device 310.

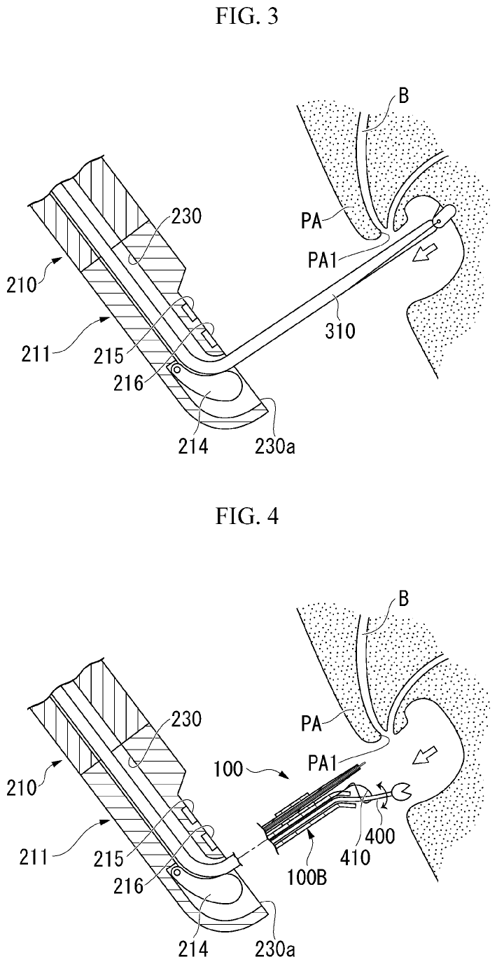

FIG. 4 is a view showing an example of the biomanipulation process.

For example, as shown in FIG. 4, the surgeon uses a grasping device-attached catheter 100B, which has grasping forceps 400 added to the catheter 100, to grasp, pull, and fix the diverticulum inner wall, causing the bile duct B to straighten and point toward an insertion shaft of the grasping device-attached catheter 100B. The grasping device-attached catheter 100B has a ring wire 410 that grasps and fixes the living organism.

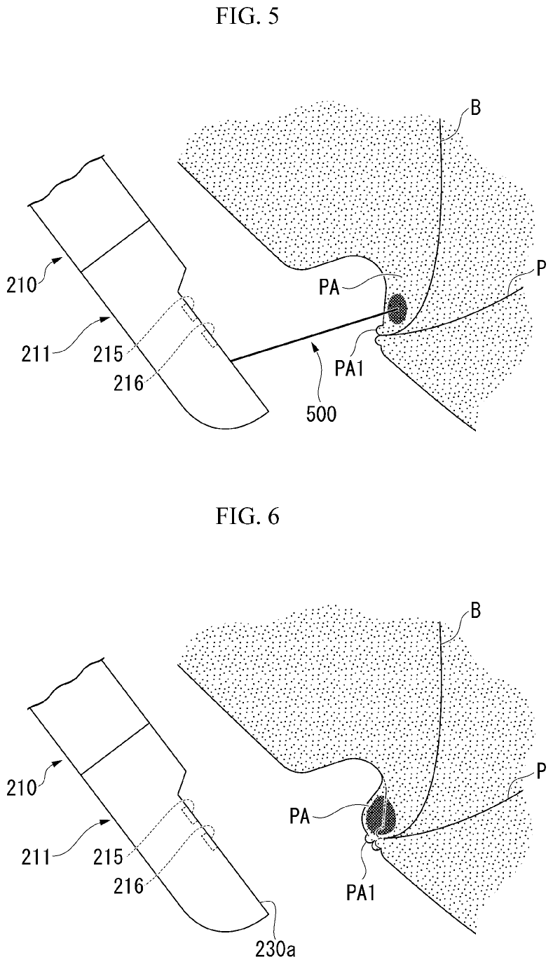

FIG. 5 and FIG. 6 are views showing an example of the biomanipulation process.

For example, as shown in FIG. 5, the surgeon uses a local injection device 500 to perform a local injection near the papilla PA, and by deforming its shape, moves the opening portion PA1 to a position visible from the endoscope 200.

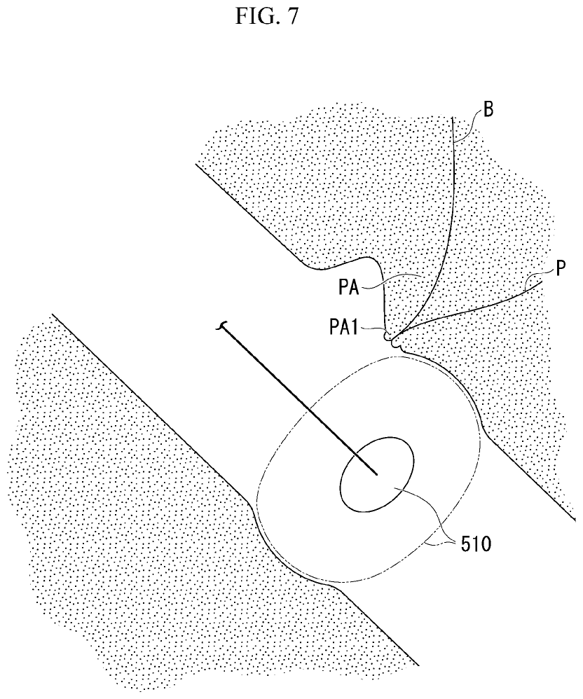

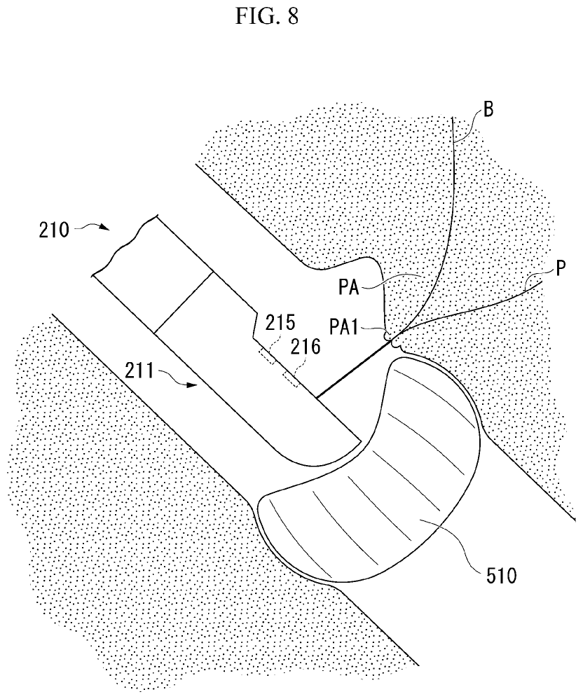

FIG. 7 and FIG. 8 are views showing an example of the biomanipulation process.

For example, as shown in FIG. 7, the surgeon expands a balloon device 510 to change the shape of the lumen, thereby moving the opening portion PA1 to a position visible from the endoscope 200.

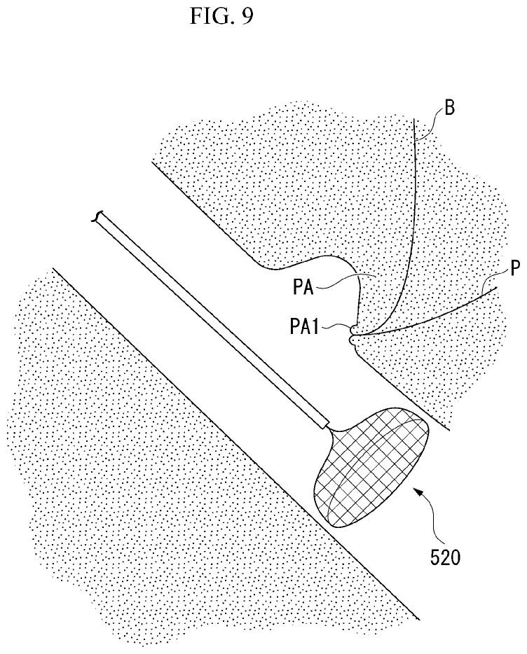

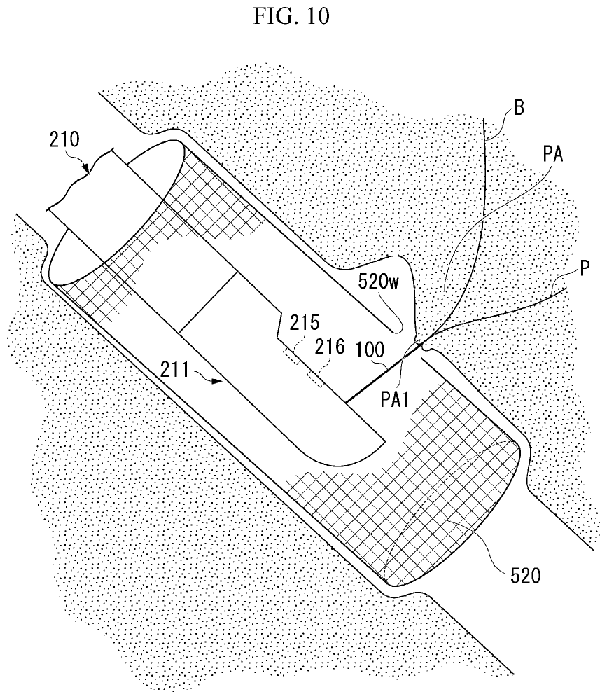

FIG. 9 and FIG. 10 are views showing an example of the biomanipulation process.

For example, as shown in FIG. 9, the surgeon places a stent 520 and changes the shape of the lumen, thereby moving the opening portion PA1 to a position visible from the endoscope 200. The stent 520 has a window 520w through which the catheter 100 can be inserted.

In addition, the suction function is used to reduce the pressure inside the placed stent, and an inner wall surface of the diverticulum is pulled out, moving the opening portion PA1 to a position visible through the endoscope 200.

If the papilla PA has high mobility, it may be difficult to direct the catheter 100 into the opening portion PA1 of the papilla PA. In this case, the surgeon makes the papilla PA difficult to move and then inserts the catheter 100 into the opening portion PA1.

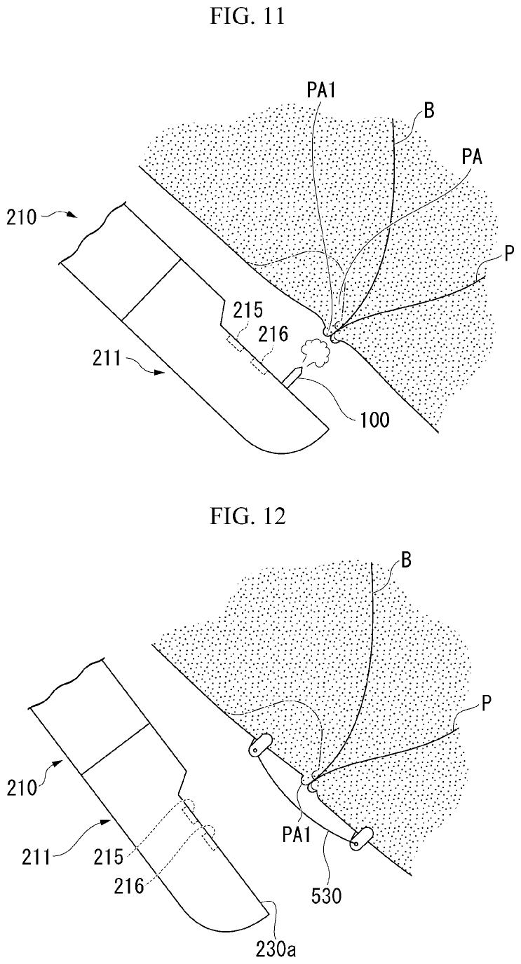

FIG. 11 is a view showing an example of the biomanipulation process.

For example, as shown in FIG. 11, the surgeon makes the papilla PA immobile by spraying cold water, salt, or a coating agent from the catheter 100 onto the opening portion PA1. Since the papilla PA becomes less likely to move, it becomes easier to insert the guide wire GW into the bile duct B. The coating agent includes, for example, sucralfate, mucoadhesive polymers, and the like.

FIG. 12 is a view showing an example of the biomanipulation process.

For example, as shown in FIG. 12, the surgeon uses a clip device 530, which has clips on both ends of a thread, to pull the tube wall until it stops moving, thereby making the papilla PA less mobile. Since the papilla PA becomes less mobile, it becomes easier to insert the guide wire GW into the bile duct B.

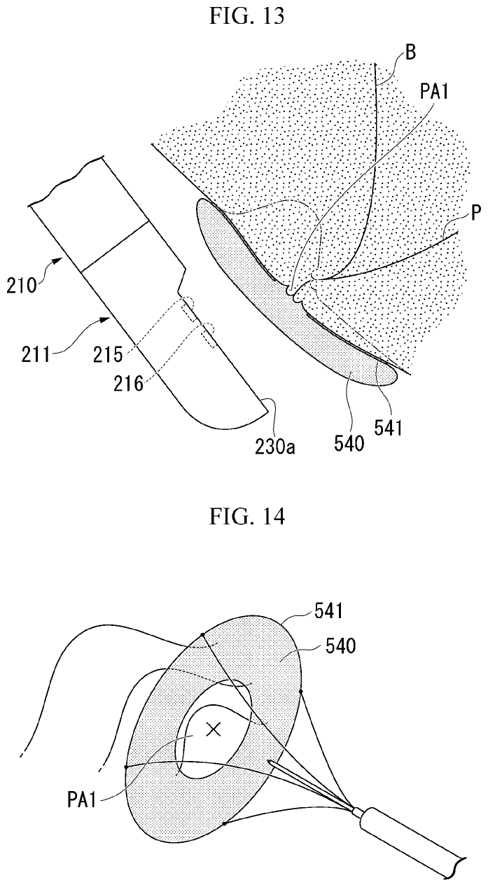

FIG. 13 and FIG. 14 are views showing an example of the biomanipulation process.

For example, as shown FIG. 13, the surgeon sprays gel 540 from the catheter 100 onto the opening portion PA1 to make the papilla PA immobile. Since the papilla PA becomes less mobile, it becomes easier to insert the guide wire GW into the bile duct B. As shown in FIG. 14, an annular gel sheet 541 may be attached to the papilla PA.

In the case of the papilla PA such as a nodular type, if the papilla PA has an obstacle PA2 such as protrusion, it may be difficult to direct the catheter 100 to the opening portion PA1 of the papilla PA. In this case, the surgeon moves the obstacle PA2 and directs the catheter 100 toward the opening portion PA1.

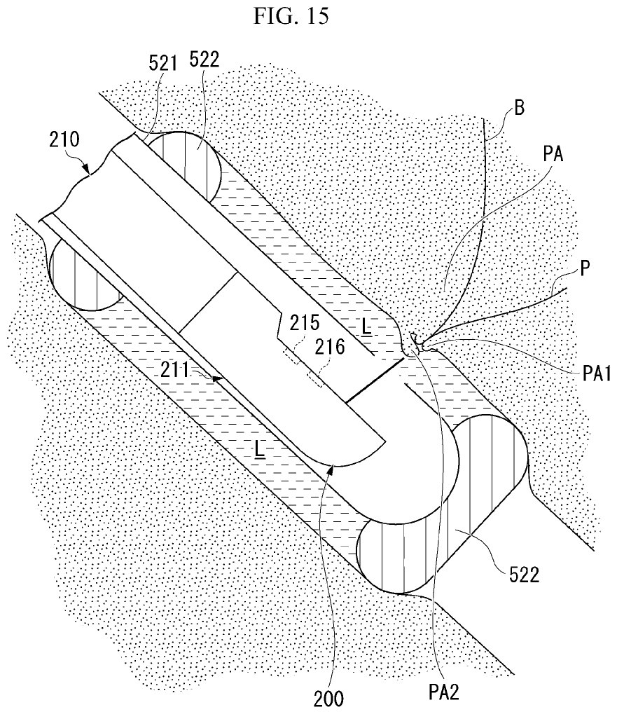

FIG. 15 is a view showing an example of the biomanipulation process.

For example, as shown in FIG. 15, the surgeon expands two balloons 522 provided on an outer tube 521 through which the endoscope 200 is inserted, and fills a space between the balloons 522 with a fluid L, thereby moving the obstacle PA2. By filling the treatment area with the fluid L, it becomes easier to observe the papilla PA, etc. By filling the space between the balloons 522 with the fluid L, the obstacle PA2 floats, and the subsequent treatment can also be carried out as usual. This process is similar to a so-called underwater procedure.

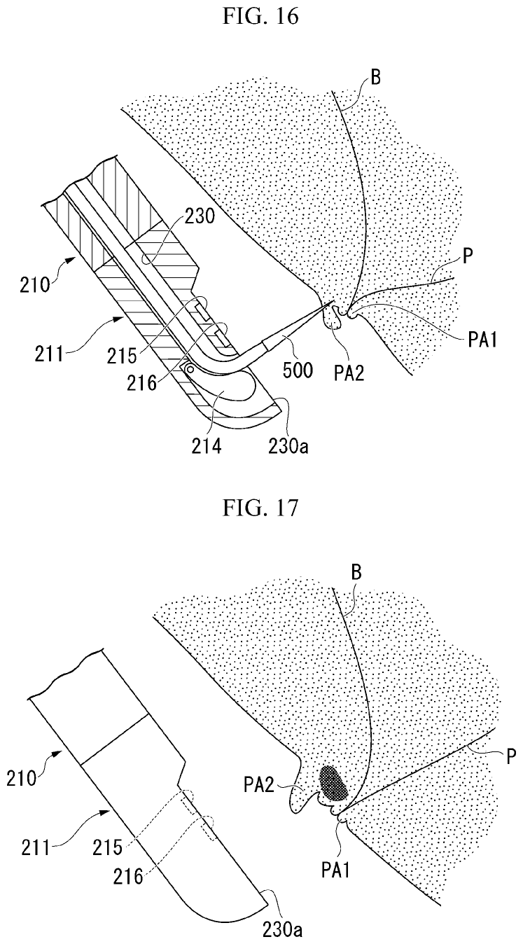

FIG. 16 and FIG. 17 are views showing an example of the biomanipulation process.

For example, as shown in FIG. 16, the surgeon uses the local injection device 500 to perform a local injection near the papilla PA, deforming its shape and thereby moving the obstacle PA2.

FIG. 18 is a view showing an example of the biomanipulation process.

For example, as shown in FIG. 18, the surgeon uses a high-frequency knife 550 to cut off at least a portion of the obstacle PA2. The surgeon may use treatment tools other than the high-frequency knife 550, such as those used for incisions of the papilla PA.

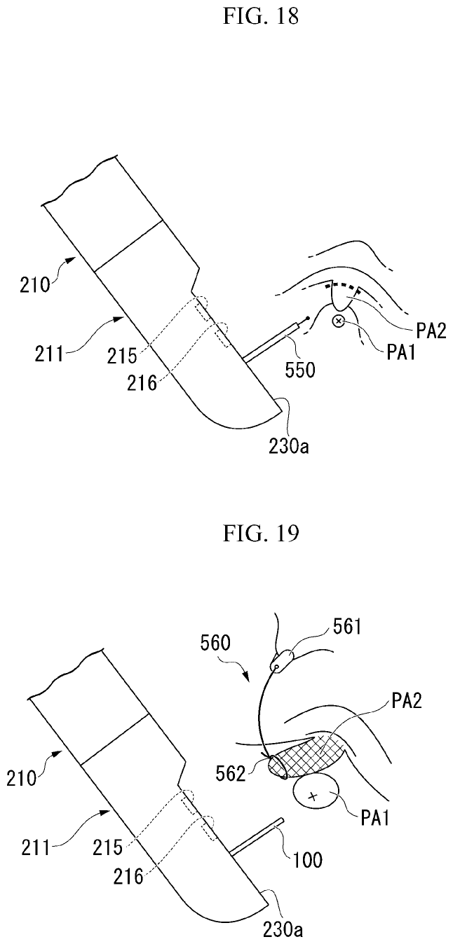

FIG. 19 is a view showing an example of the biomanipulation process.

For example, as shown in FIG. 19, the surgeon moves the obstacle PA2 using a fixing device 560 having a clip 561 at one end of the thread and a snare 562 at the other end. Specifically, the surgeon hooks the snare 562 to the obstacle PA2 and fixes the clip 561 to the tube wall.

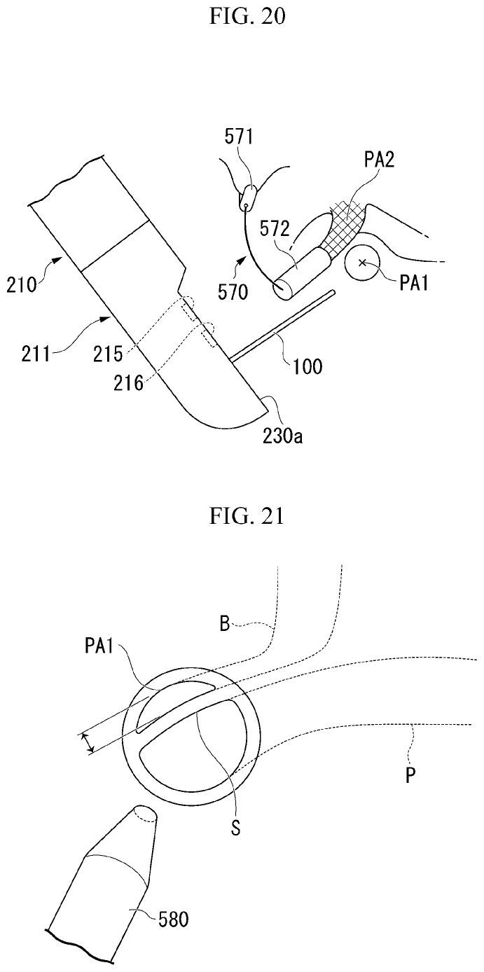

FIG. 20 is a view showing an example of the biomanipulation process.

For example, as shown in FIG. 20, the surgeon moves the obstacle PA2 using a fixing device 570 with a clip 571 at one end of the thread and a cap 572 at the other end. Specifically, the surgeon hooks the cap 572 onto the obstacle PA2 and fixes the clip 571 to the tube wall.

In the papilla PA of the septal type, the septum S between the bile duct B and the pancreatic duct P is unclear, making it difficult to identify the opening portion PA1. In this case, the surgeon widens the opening portion PA1.

For example, the surgeon widens the opening portion PA1 by feeding the subject food. It is desirable that the food contains a lot of fat. By enlarging the opening portion PA1, the angle range of the guide wire GW that can be inserted can be expanded.

For example, the surgeon widens the opening portion PA1 by incising the opening portion PA1. The surgeon may widen the opening portion PA1 by pre-cutting the pancreatic duct. The surgeon may widen the opening portion PA1 by incising the septum S between the bile duct B and the pancreatic duct P.

FIG. 21 is a view showing an example of the biomanipulation process.

For example, as shown in FIG. 21, the surgeon may widen the opening portion PA1 of the bile duct B by narrowing the opening of the pancreatic duct P by suctioning air within the pancreatic duct P with a suction device 580.

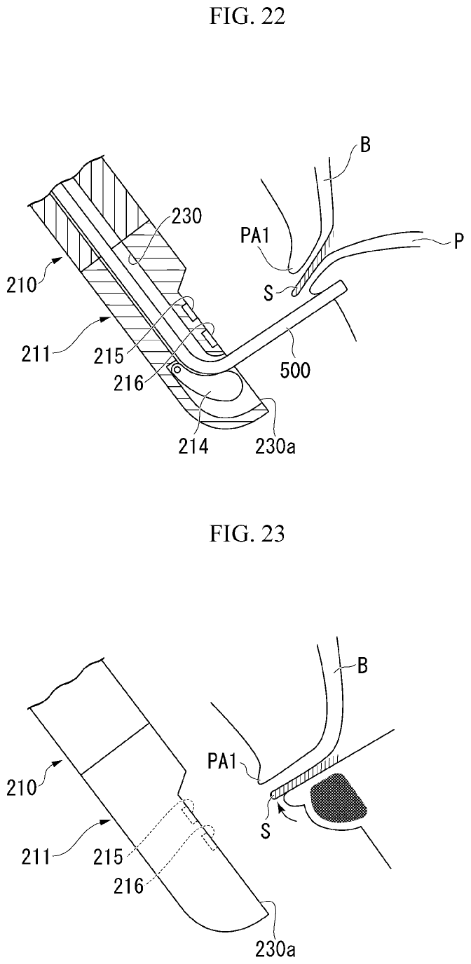

FIG. 22 and FIG. 23 are views showing an example of the biomanipulation process.

For example, as shown in FIG. 22, the surgeon uses the local injection device 500 to perform a local injection near the papilla PA, deforming its shape and widening the opening portion PA1.

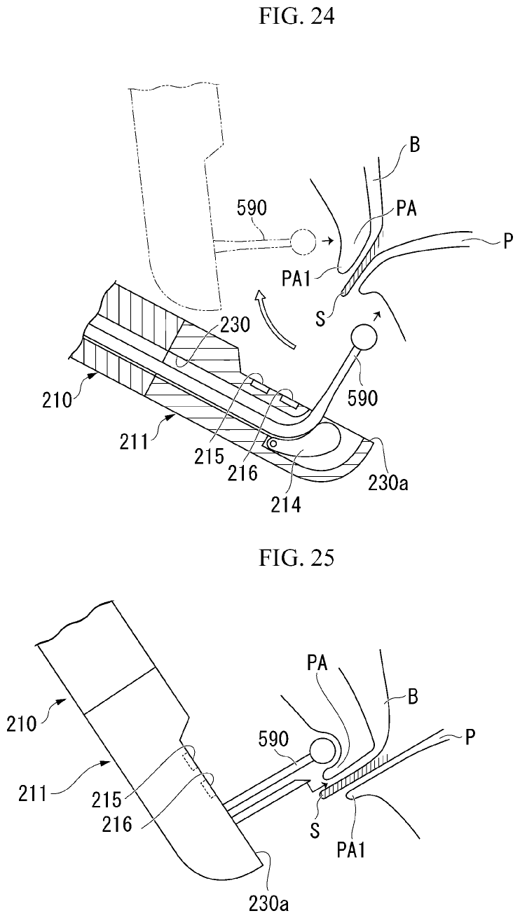

FIG. 24 and FIG. 25 are views showing an example of the biomanipulation process.

For example, as shown in FIG. 24, the surgeon uses a pressing device 590 to adjust the pressing angle and pressing area relative to the septum S, thereby widening the opening portion PA1. It is desirable for the surgeon to pinch the opening portion PA1 of the bile duct B and press both sides with the pressing device 590.

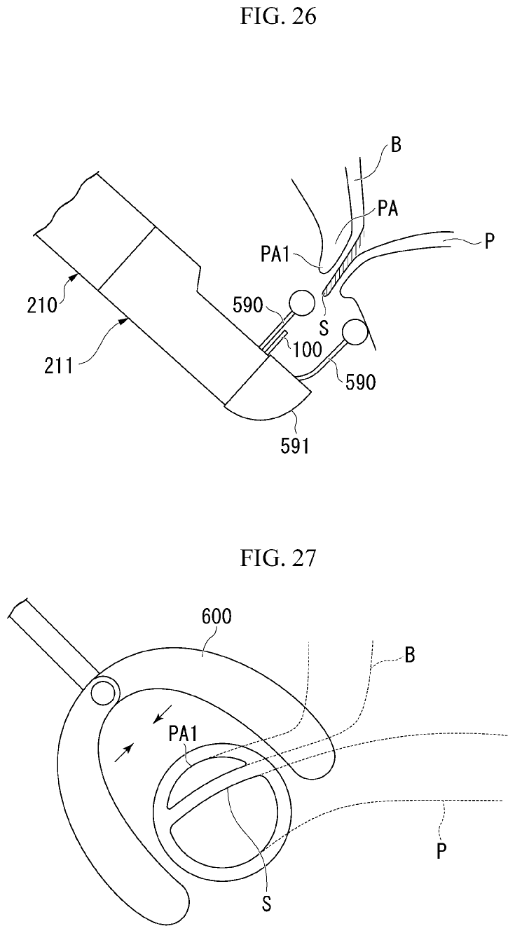

FIG. 26 is a view showing an example of the biomanipulation process.

For example, as shown in FIG. 26, at least one of the pressing devices 590 may be provided on a cap 591 that is removably attached to the tip portion 211 of the endoscope 200.

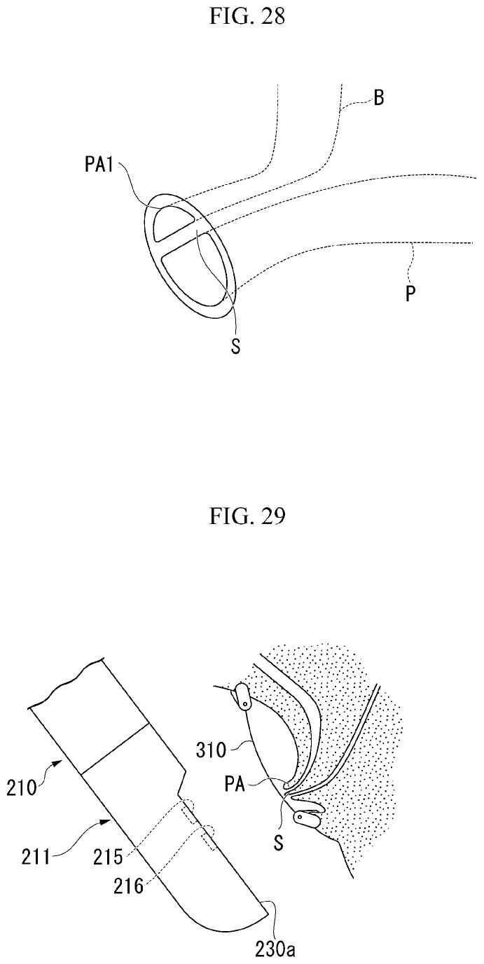

FIG. 27 and FIG. 28 are views showing an example of the biomanipulation process.

For example, as shown in FIG. 27, the surgeon uses a clamping device 600 to clamp the papilla PA, thereby widening the opening portion PA1 of the bile duct B.

In the papilla PA of the septal type or the like, the septum S between the bile duct B and the pancreatic duct P is unclear, making it difficult to identify the opening portion PA1. In this case, the surgeon acts on the living organism, loosening the angle of the bile duct course relative to the duodenum course.

FIG. 29 is a view showing an example of the biomanipulation process.

For example, as shown in FIG. 29, the surgeon uses the threaded clip device 310 to pull the entrance of the bile duct B and the entrance of the pancreatic duct P to opposite sides, pushing and pulling the papilla PA and its surroundings. By strongly pulling the entrance of the bile duct B, which is the insertion target, the insertion angle for inserting the guide wire GW or the like into the bile duct B can be loosened. Meanwhile, the insertion angle for inserting the guide wire GW or the like into the pancreatic duct P, which is not the insertion target, is tight, which prevents the guide wire GW or the like from being inserted into the pancreatic duct P by mistake.

The surgeon uses the pressing device 590 to push up the small intestine side of the papilla PA, that is, to push up the pancreatic duct P toward the stomach, thereby loosening the insertion angle for inserting the guide wire GW or the like into the bile duct B.

### <Step S4: Bile Duct Intubation Process>

In step S4, the surgeon brings the tip of the catheter 100, which has the guide wire GW pre-loaded, close to the opening portion PA1 of the bile duct B of the papilla PA. Alternatively, the tip of the catheter 100 is slightly inserted into the opening portion PA1 of the bile duct B. The guide wire GW is extended along the expected bile duct course, and the guide wire GW is inserted into the bile duct B. The catheter 100 is inserted into the bile duct B along the guide wire GW.

In this process, a sphincterotome may be used instead of the catheter 100.

In addition, the catheter 100 may be intubated into the bile duct B without protruding the guide wire GW.

### <Step S5: Contrast Process>

In step S5, the surgeon injects a contrast agent C into the syringe 53, causing the contrast agent C to flow into the bile duct B through the catheter 100. The surgeon performs X-ray photography to obtain an X-ray image showing the bile duct B, etc.

### <Step S6: Guide Wire Indwelling Process>

In step S6, the surgeon removes the catheter 100 while leaving the guide wire GW in the bile duct B. Accordingly, only the guide wire GW is placed in the bile duct B, creating an access route to the bile duct from the inside of the duodenum.

According to the insertion method into the bile duct B of the embodiment, the guide wire GW can be easily inserted into the bile duct. In addition, the guide wire GW inserted into the bile duct makes it easy to an insert medical device such as a stent delivery device or the like into the bile duct.

Hereinabove, the first embodiment has been described in detail with reference to the drawings, but the specific configuration is not limited to this embodiment, and design changes that do not deviate from the spirit of the present disclosure are also included. In addition, the components shown in the above-mentioned embodiment and variant can be configured in any suitable combination.

### Second Embodiment

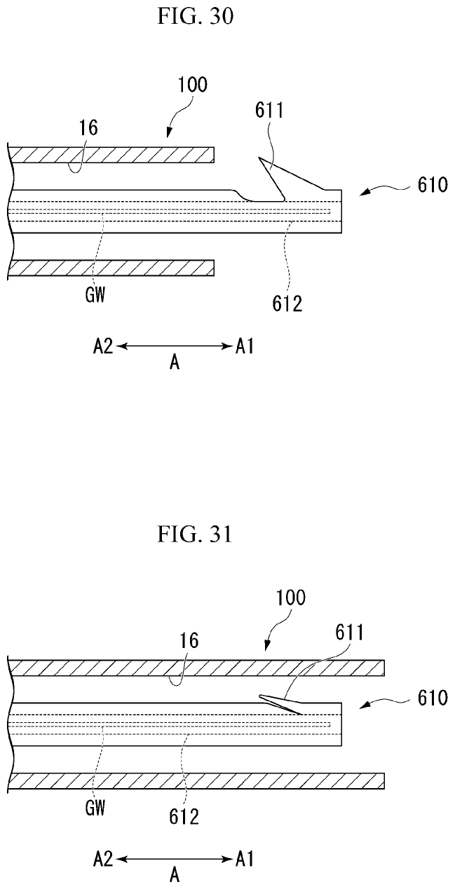

A cannulation method according to a second embodiment will be described with reference to FIG. 30 to FIG. 49. In the following description, for configurations that are common to those already described, the same reference signs will be used and duplicate descriptions will be omitted.

Actions and effects of the endoscope treatment system 300 according to the embodiment will be described. Specifically, a procedure for treating the bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S4: Bile Duct Intubation Process>

In the case of the papilla PA, such as long protrusion, the bile duct B is long and the route is meandering. In this case, the surgeon grasps and pulls the papilla and the area near the papilla to straighten the bile duct B, and then intubates the course of the bile duct B in line with the direction of advance of the intubation device.

FIG. 30 to FIG. 33 are views showing an example of a bile duct intubation process.

For example, as shown in FIG. 30 and FIG. 31, the surgeon uses a hook device 610 that can be inserted through the guide wire lumen 16 of the catheter 100. The hook device 610 has a hook 611 at its tip that faces the base end side. The hook device 610 is provided with a lumen 612 through which the guide wire GW can be inserted. The hook device 610 is receivable in the guide wire lumen 16, as shown in FIG. 31.

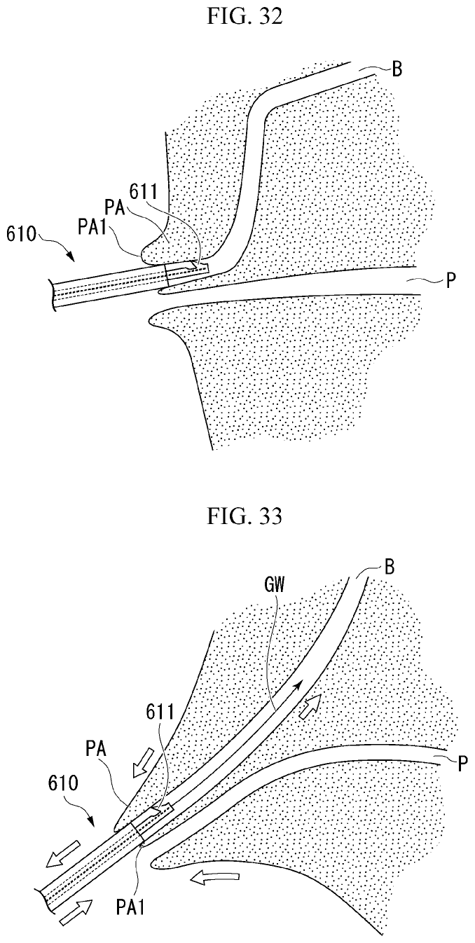

As shown in FIG. 32, the surgeon slightly inserts the tip opening 12 of the catheter 100 into the opening portion PA1 of the papilla PA, causing the hook device 610 to protrude. As shown in FIG. 33, the surgeon hooks the hook 611 to the inside of the papilla PA and pulls the papilla PA. The bile duct B is deformed into a straight line. The surgeon pulls the papilla PA with the hook 611, pushes out the guide wire GW, and inserts the guide wire GW into the bile duct B. Since the bile duct B was deformed into a straight line, the surgeon is able to smoothly insert the guide wire GW into the bile duct B. The surgeon may use a high-frequency device to an incision part of the papilla PA while pulling the papilla PA. The hook device 610 may have a tip portion with a tendency to bend to make it easier to hook onto the papilla PA.

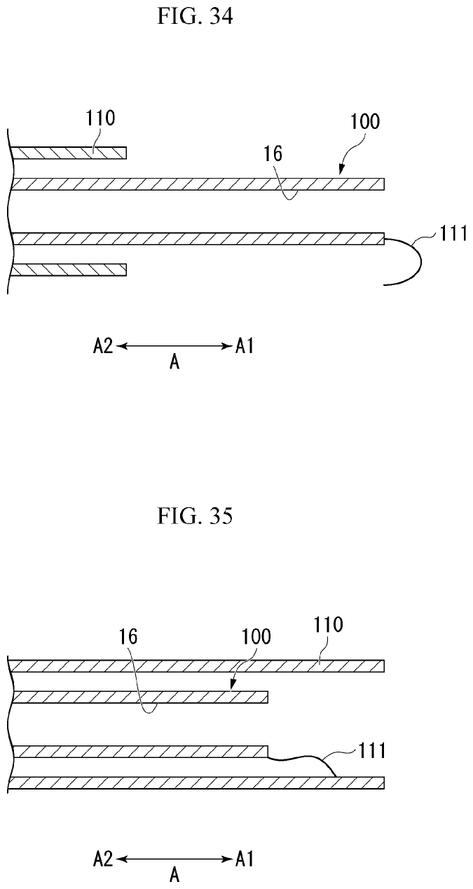

FIG. 34 and FIG. 35 are views showing an example of the bile duct intubation process.

For example, as shown in FIG. 34 and FIG. 35, the surgeon may use the catheter 100 having a hook 111 at the tip. The surgeon hooks the hook 111 and pulls the papilla PA. As shown in FIG. 35, the hook 111 may be received in an outer sheath 110 disposed on the outside of the catheter 100 before use. The hook 111 may have conductivity. In this case, the hook 111 is connected to a high frequency power supply by a wire or the like. The surgeon can incise a part of the papilla PA while pulling the papilla PA.

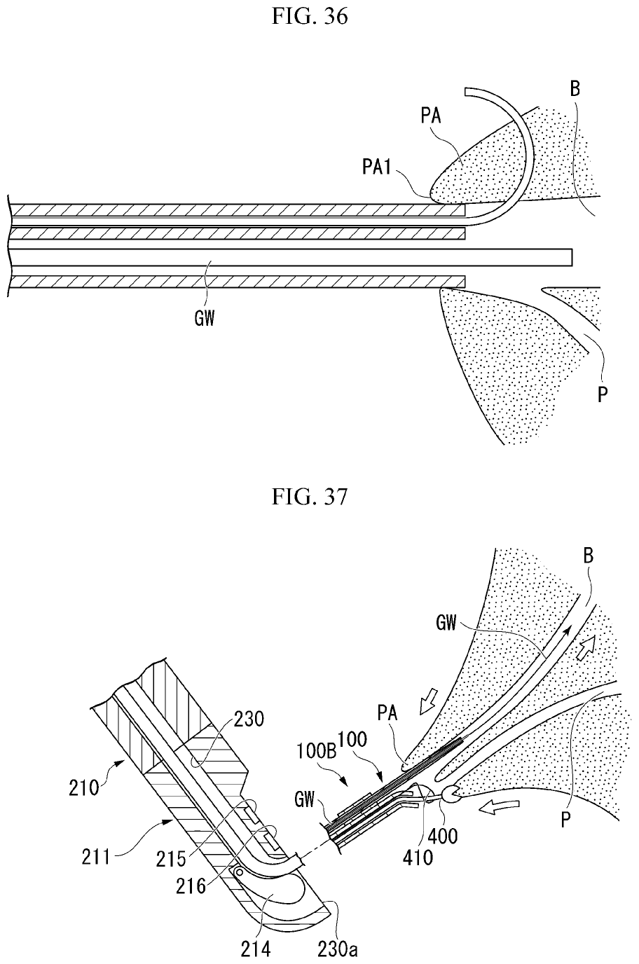

FIG. 36 is a view showing an example of the bile duct intubation process.

For example, as shown in FIG. 36, the surgeon inserts a hook device 620 into one lumen of the catheter 100, which has two lumens. The tip portion of the hook device 620 has a loop-shaped tendency to bend. The surgeon protrudes the tip portion of the hook device 620 while the tip of the catheter 100 is slightly inserted into the papilla PA, and penetrates the tip portion of the hook device 620 from the inside of the papilla PA to the duodenum side. The surgeon pulls the hook device 620 to pull the papilla PA. The surgeon passes the guide wire GW through the other lumen and inserts the guide wire GW into the bile duct B. Since the bile duct B is deformed into a straight line, the surgeon is able to smoothly insert the guide wire GW into the bile duct B.

The hook device 620 may have conductivity. In this case, the hook device 620 is connected to a high frequency power supply. The surgeon can incise a part of the papilla PA while pulling the papilla PA. It is safe because the tissue is under tension and the cutting is done from the inside of the papilla PA. The hook device 620 can also be used as a substitute for a high-frequency knife.

FIG. 37 is a view showing an example of the bile duct intubation process.

For example, as shown in FIG. 37, the surgeon uses the grasping forceps of the grasping device-attached catheter 100B to pull the area near the papilla, deforming the bile duct B into a straight line. Since the bile duct B is deformed into a straight line, the surgeon is able to smoothly insert the guide wire GW into the bile duct B.

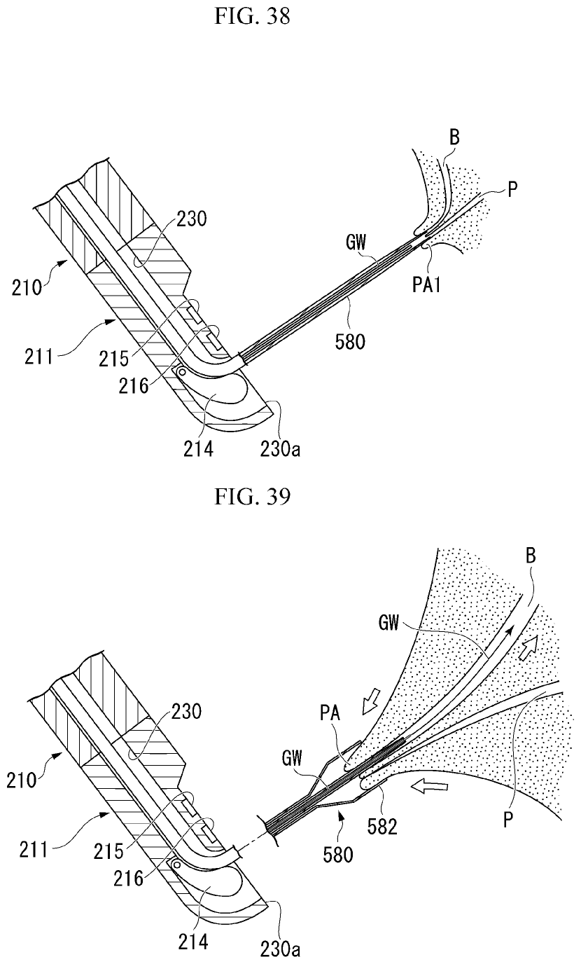

FIG. 38 and FIG. 39 are views showing an example of the bile duct intubation process.

For example, as shown in FIG. 38, the surgeon pulls the area near the papilla with the suction device 580, deforming the bile duct B into a straight line. The surgeon inserts the guide wire GW, through which the suction device 580 has been passed, into the bile duct B. Since the bile duct B is deformed into a straight line, the surgeon is able to smoothly insert the guide wire GW into the bile duct B. As shown in FIG. 39, the suction device 580 may include a suction hood 582. The suction hood 582 has a wide tip, allowing for suction of a wide range of the papilla PA. The suction hood 582 may have barbs that hook onto the papilla PA.

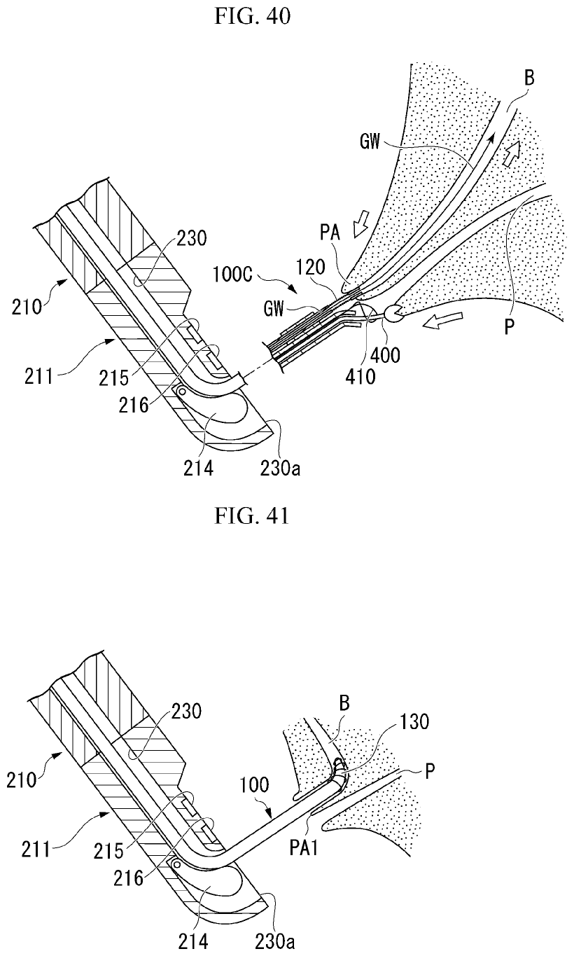

FIG. 40 and FIG. 41 are views showing an example of the bile duct intubation process.

For example, as shown in FIG. 40, the surgeon uses a grasping forceps of a grasping device-attached catheter 100C to pull the area near the papilla, deforming the bile duct B into a straight line. Compared to the grasping device-attached catheter 100B, the grasping device-attached catheter 100C further has a bellows-shaped curved section 120 at the tip portion of the catheter 100. The bending of the curved section 120 allows the surgeon to insert the guide wire GW from the catheter 100 into the bile duct B even if the bile duct B is not completely deformed into a straight line.

In addition, as shown in FIG. 41, the surgeon inserts the catheter 100, which has a screw-shaped tip portion 130, into the bile duct B while rotating the catheter 100. Even if the bile duct B has a portion with a large bending angle, the catheter 100 can be moved to the tip side by screwing in the rotated tip portion 130, allowing intubation.

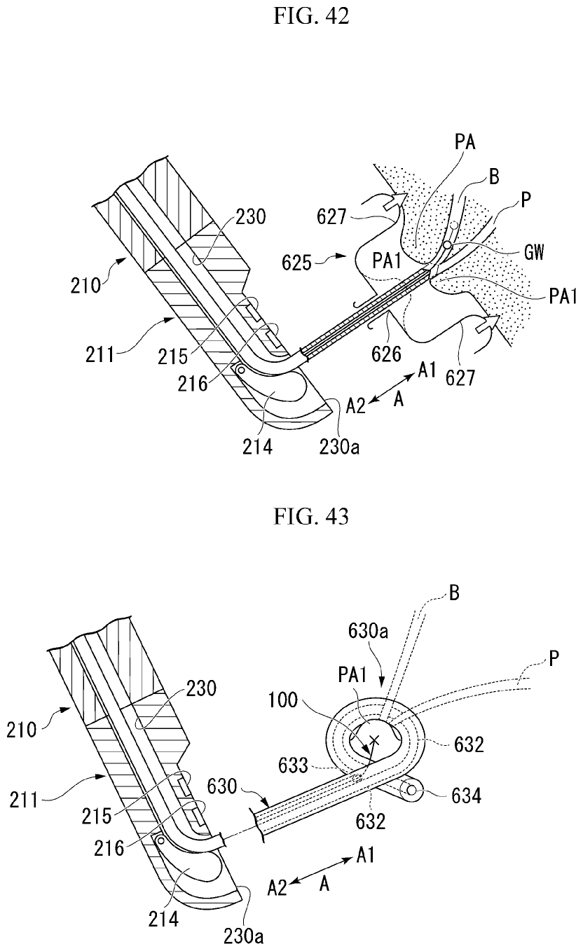

FIG. 42 is a view showing an example of the bile duct intubation process.

For example, as shown in FIG. 42, the surgeon uses a tubular device 625 made of woven wire. The tubular device 625 has a narrow tube portion 626 in its center portion through which the catheter 100 can be inserted. The narrow tube portion 626 has elasticity and provides adequate pressure to the inserted catheter 100. A tip portion 627 of the tubular device 625 has a larger inner diameter and is dome-shaped so that it can be placed over at least a portion of the papilla PA. When the surgeon loads the guide wire GW up to the tip of the catheter 100 and pushes the catheter 100 into the tip side A1, the narrow tube portion 626 pushes the tip portion 627 into the tip side, causing the tip portion 627 to deform. The tip portion of the catheter 100 is inserted into the opening portion PA1 of the papilla PA. After that, when the surgeon releases the catheter 100 and releases the pushing force, the catheter 100 is pulled back to the base end side A2. Here, the papilla PA is pulled to the base end side A2 by the catheter 100. As the surgeon repeats this operation, the papilla PA is pulled into the tip portion 627 of the tubular device 625. As a result, the bile duct B is deformed into a straight line. Since the bile duct B is deformed into a straight line, the surgeon is able to smoothly insert the guide wire GW into the bile duct B.

FIG. 43 is a view showing an example of the bile duct intubation process.

For example, as shown in FIG. 43, the surgeon uses a guiding sheath 630. The guiding sheath 630 has a loop-shaped tip portion 630a such as a big tail type. The guiding sheath 630 has a catheter lumen 631 through which the catheter 100 can be inserted, and a suction lumen 632. The catheter lumen 631 communicates with an opening 633 provided on the base end side A2, rather than the loop-shaped tip portion 630a. The suction lumen 632 communicates with a suction opening 634 provided at the tip of the guiding sheath 630. The surgeon fits the loop-shaped tip portion 630a into the papilla PA and presses the tip portion 630a against the tube wall. Next, the surgeon performs suction from the suction lumen 632 and the suction opening 634 to fix the tip portion 630a to the tube wall. The surgeon pulls the papilla PA by pulling the guiding sheath 630 toward the base end side A2, and the bile duct B is deformed into a straight line. Since the bile duct B is deformed into a straight line, the surgeon is able to smoothly insert the guide wire GW into the bile duct B.

In the case of the papilla PA, such as a long protrusion, the bile duct B is long and the route is meandering. In this case, the surgeon intubates the curved portion of the bile duct B, straightening the curved portion each time the object passes therethrough.

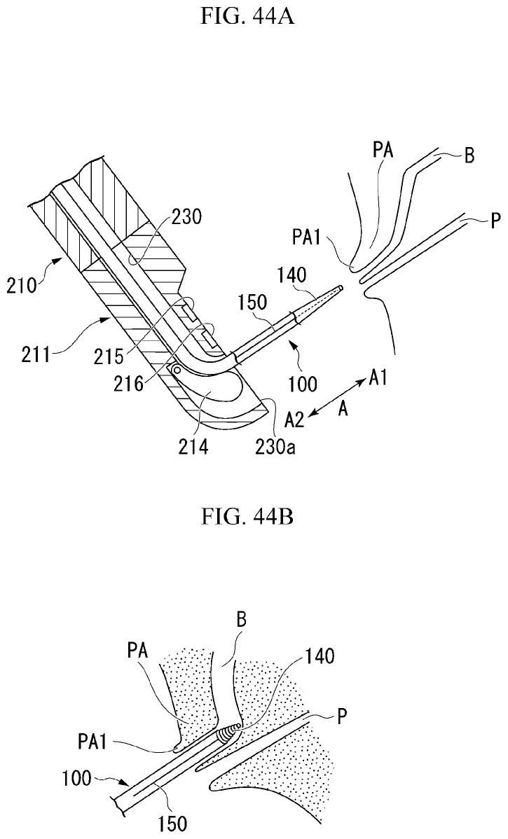

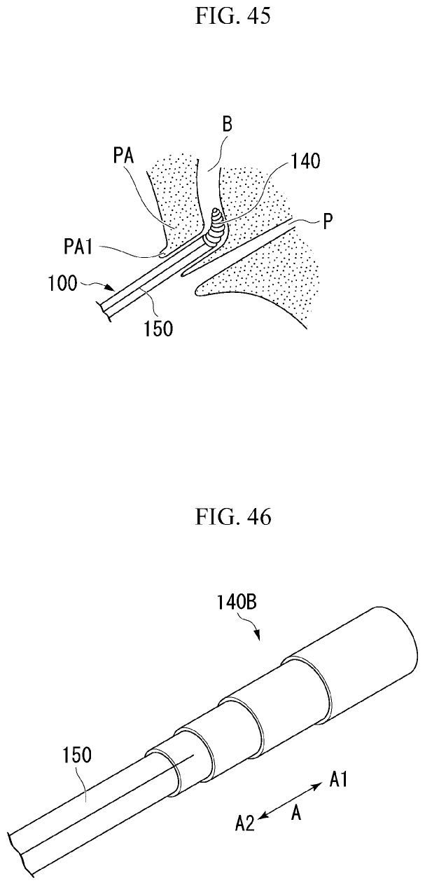

FIG. 44A, FIG. 44B and FIG. 45 are views showing an example of the bile duct intubation process.

For example, as shown in FIG. 44B, the surgeon uses the catheter 100 including a soft tip portion 140. As shown in FIG. 44, the surgeon inserts the tip portion 140 into the bile duct B. When the tip portion 140 hits the curved portion of the bile duct B, the tip portion 140 is collapsed in the longitudinal direction and shortened. The surgeon inserts a rigid core rod 150 into the guide wire lumen 16 and extends the collapsed tip portion 140 along the longitudinal direction A. Here, as shown in FIG. 45, the core rod 150 is slightly curved to follow the bile duct B due to the resistance received from the tip portion 140. The bile duct B deforms to fit the curved core rod 150. As a result, the curved portion of the bile duct B becomes less curved. Since the bile duct B is gently curved, the surgeon can smoothly insert the guide wire GW into the bile duct B.

FIG. 46 is a view showing a tip portion 140B, which is a variant of the tip portion 140.

The tip portion 140B is formed into a tubular shape by wrapping a flexible thin sheet around it. The tip portion 140B can be stretched in the longitudinal direction A by changing the degree of winding of the sheet. The tip portion 140B functions similarly to the tip portion 140.

In the case of the papilla PA, such as a long protrusion or the like, the bile duct B is long and the route is meandering. In this case, the surgeon uses a device to pull the tube wall backward and intubates the bile duct B while pulling the bile duct B.

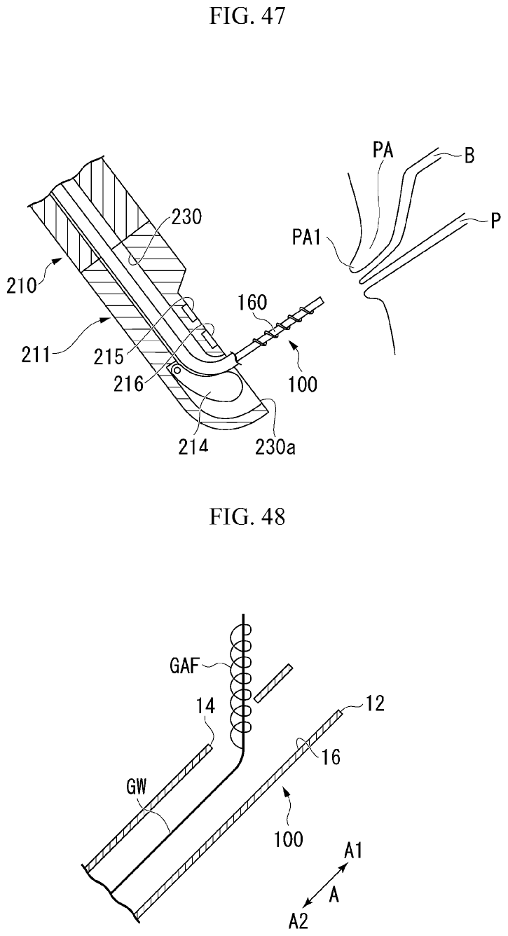

FIG. 47 is a view showing an example of the bile duct intubation process.

For example, as shown in FIG. 47, the catheter 100 including a spiral fin 160 is used. The surgeon inserts the catheter 100 into the bile duct B while rotating the spiral fin 160, so that even if the bile duct B has a curved portion, the surgeon can insert the catheter 100 into the bile duct B while pulling the bile duct B toward the surgeon. The guide wire GW through which the catheter 100 is inserted may have an imaging function. The surgeon can use the imaging function of the guide wire GW to observe the inside of the bile duct B and align the tip of the catheter 100 with the opening portion PA1.

FIG. 48 is a view showing an example of the bile duct intubation process.

For example, as shown in FIG. 48, the guide wire GW including a spiral fin GWF is used. The catheter 100 has a side opening 14, and the guide wire GW with the spiral fin GWF can also protrude from the side opening 14. The surgeon can change the protrusion direction of the guide wire GW protruding from the side opening 14 by rotating the catheter 100. By inserting the guide wire GW into the bile duct B while rotating the spiral fin GWF, the surgeon can insert the guide wire GW into the bile duct B while pulling it in the bile duct B, even if the bile duct B has the curved portion.

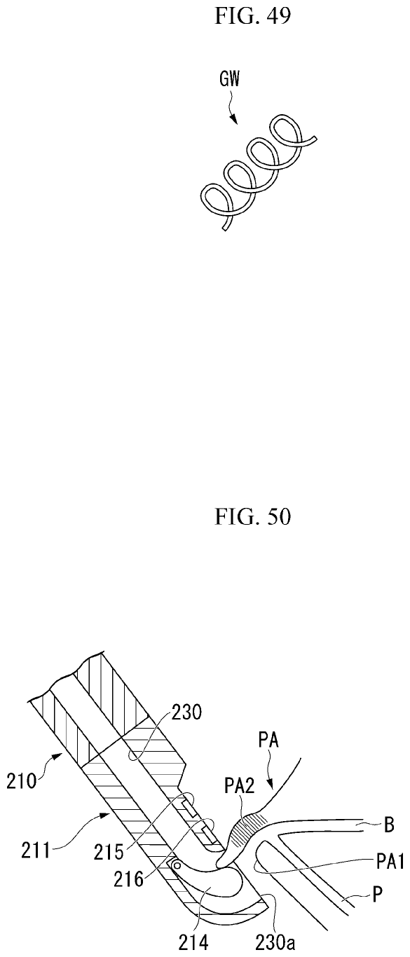

FIG. 49 is a view showing an example of the bile duct intubation process.

For example, as shown in FIG. 49, the guide wire GW formed in a spiral shape is used. The surgeon can insert the guide wire GW into the bile duct B while rotating the guide wire GW, and can insert the guide wire GW into the bile duct B while pulling the bile duct B together, even if the bile duct B has the curved portion.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, the second embodiment has been described in detail with reference to the drawings, but the specific configuration is not limited to this embodiment, and design changes that do not deviate from the spirit of the present disclosure are also included. In addition, the components shown in the above-mentioned embodiment and variant can be configured in any suitable combination.

### Third Embodiment

A cannulation method according to a third embodiment will be described with reference to FIG. 50 to FIG. 57. In the following description, for configurations that are common to those already described, the same reference signs will be used and duplicate descriptions will be omitted.

Actions and effects of the endoscope treatment system 300 according to the embodiment will be described. Specifically, a procedure for treating the bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S3C: Biomanipulation Process>

The papilla PA, such as a nodular type or the like, may have the obstacle PA2, such as a protrusion or the like. In this case, the surgeon inserts the catheter 100 or the guide wire GW while avoiding the obstacle PA2.

FIG. 50 is a view showing an example of the biomanipulation process.

For example, as shown in FIG. 50, the surgeon pulls in the obstacle PA2 by suctioning the obstacle PA2 from a channel 230, and inserts the guide wire GW into the bile duct B. The surgeon can remove the obstacle PA2 from near the opening portion PA1 and insert the guide wire GW into the bile duct B. In addition, the papilla PA can also be fixed. Further, the opening portion PA1 can also be oriented toward the endoscope 200.

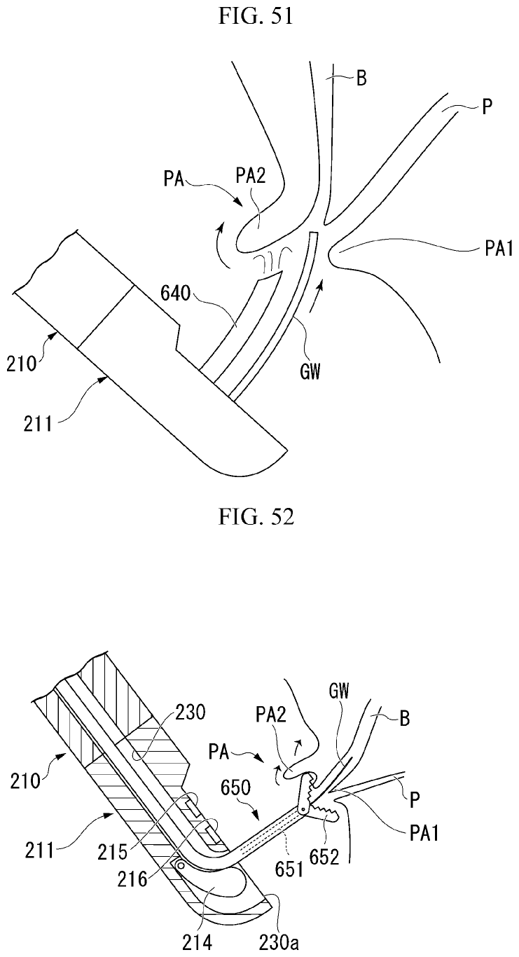

FIG. 51 is a view showing an example of the biomanipulation process.

For example, as shown in FIG. 51, the surgeon uses an air and water supply catheter 640 to supply air or water to the obstacle PA2 to move the obstacle PA2, and then inserts the guide wire GW into the bile duct B. The surgeon can remove the obstacle PA2 from near the opening portion PA1 and insert the guide wire GW into the bile duct B.

FIG. 52 is a view showing an example of the biomanipulation process.

For example, as shown in FIG. 52, the surgeon uses a guide sheath 650 to move the obstacle PA2 and insert the guide wire GW into the bile duct B. The guide sheath 650 has a lumen 651 through which the guide wire GW can be inserted, and an opening/closing portion 652 provided at the tip. The surgeon inserts the opening/closing portion 652 under the obstacle PA2 to open the opening/closing portion 652, thereby moving the obstacle PA2 outward and inserting the guide wire GW into the bile duct B. The surgeon can remove the obstacle PA2 from near the opening portion PA1 and insert the guide wire GW into the bile duct B. The guide sheath 650 may have a tendency to bend. The opening/closing portion 652 may be self-scaling.

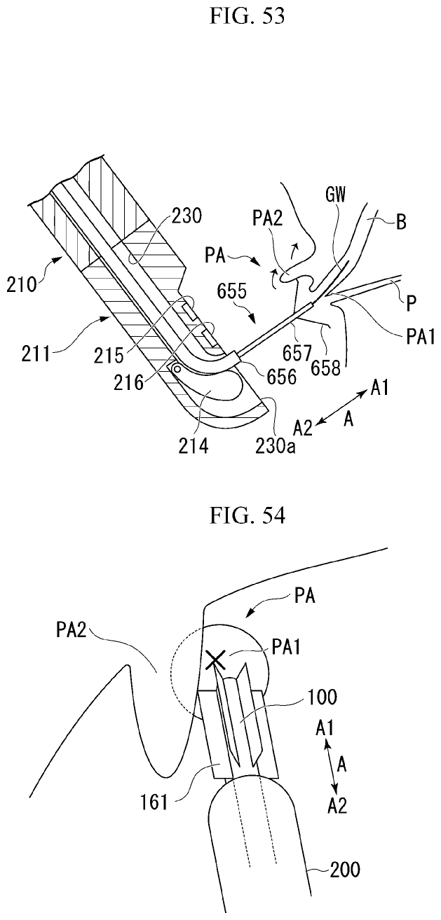

FIG. 53 is a view showing an example of the biomanipulation process.

For example, as shown in FIG. 53, the surgeon uses a guide sheath 655 to move the obstacle PA2 and insert the guide wire GW into the bile duct B. The guide sheath 655 has an outer sheath 656, a sheath 657 through which the outer sheath 656 is inserted so as to be able to move back and forth, and an opening/closing portion 658 provided at the tip of the sheath 657. The opening/closing portion 658 is self-scaling, and expands outward as the sheath 657 protrudes from the outer sheath 656 to the tip side A1. The surgeon inserts the sheath 657 under the obstacle PA2 to open the opening/closing portion 658, thereby moving the obstacle PA2 outward and inserting the guide wire GW into the bile duct B. The opening/closing portion 658 is self-scaling, which simplifies the surgeon's manipulation.

FIG. 54 is a view showing an example of the biomanipulation process.

For example, as shown in FIG. 54, the surgeon uses the catheter 100 including a fin 161. The fin 161 extends in the longitudinal direction A. The surgeon rotates the catheter 100 to move the obstacle PA2 with the fin 161, and inserts the guide wire GW into the bile duct B. The surgeon can remove the obstacle PA2 from near the opening portion PA1 and insert the guide wire GW into the bile duct B.

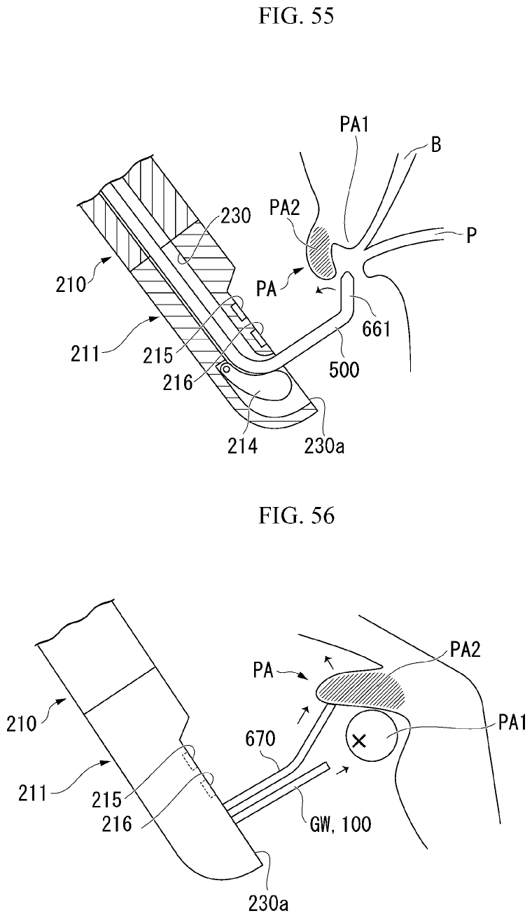

FIG. 55 is a view showing a view showing an example of the biomanipulation process.

For example, as shown in FIG. 55, the surgeon uses a curving device 660. The curving device 660 has a tip portion 661 that is actively bendable. The surgeon bends the tip portion 661 to move the obstacle PA2 and inserts the curving device 660 into the bile duct B. The curving device 660 may be the catheter 100 or the guide wire GW. The tip portion 661 may be a passively bending member rather than an actively bending member. In this case, the tip portion 661 may have a tendency to bend.

FIG. 56 is a view showing an example of the biomanipulation process.

For example, as shown in FIG. 56, the surgeon uses a push-aside device 670 to move the obstacle PA2 and insert the catheter 100 or the guide wire GW into the bile duct B. The push-aside device 670 and the device inserted into the bile duct B are separate, simplifying the surgeon's operation.

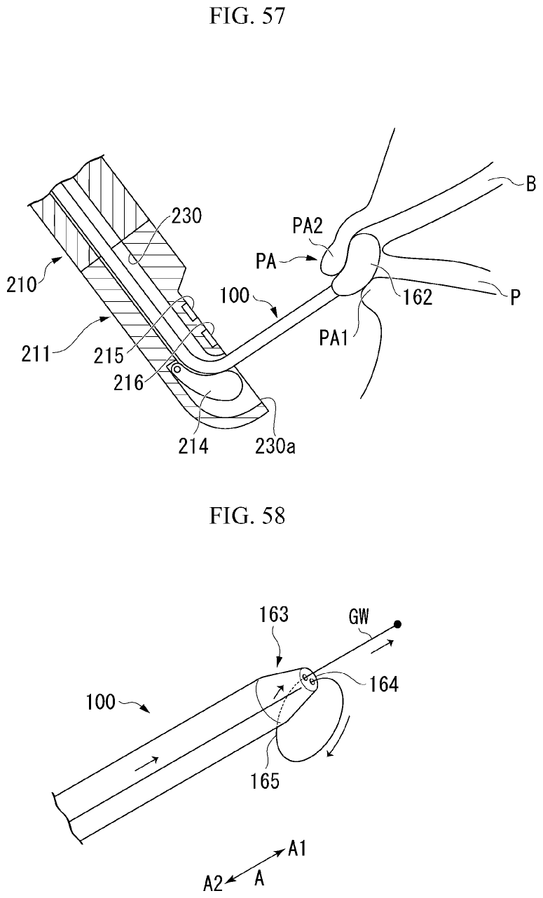

FIG. 57 is a view showing an example of the biomanipulation process.

For example, as shown in FIG. 57, the surgeon uses the catheter 100 including a balloon 162. The surgeon expands the balloon 162, moves the obstacle PA2 with the balloon 162, and inserts the guide wire GW into the bile duct B. The surgeon can remove the obstacle PA2 from near the opening portion PA1 and insert the guide wire GW into the bile duct B.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, the third embodiment has been described in detail with reference to the drawings, but the specific configuration is not limited to this embodiment, and design changes within the scope that do not deviate from the spirit of the present disclosure are also included. In addition, the components shown in the above-mentioned embodiment and variant can be configured in any suitable combination.

### Fourth Embodiment

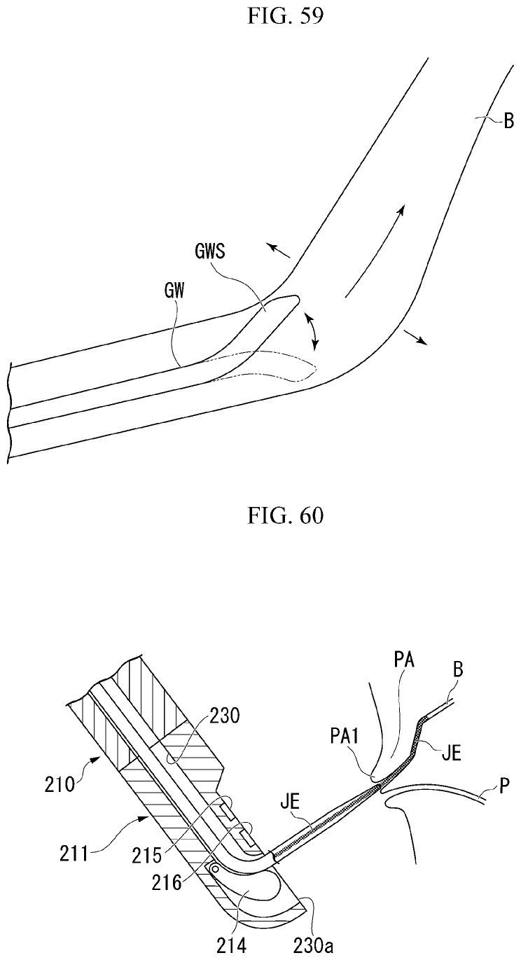

A cannulation method according to a fourth embodiment will be described with reference to FIG. 58 and FIG. 59. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

<step S3D: Biomanipulation Process>

In the case of the papilla PA, such as a long protrusion, intradiverticular papillae, or the like, the surgeon inserts an insertion device while restoring the collapsed conduit to its original shape.

FIG. 58 is a view showing an example of the biomanipulation process.

For example, as shown in FIG. 58, the surgeon uses the catheter 100 including a tip portion 163 that can hold the guide wire GW in a loop shape. The tip portion 163 has a tip opening 164 and a side opening 165 through which the guide wire GW can be inserted. The guide wire GW passes through the tip opening 164 and the side opening 165 and protrudes from the tip opening 164 to the tip side A1. As the surgeon pushes the guide wire GW into the tip side A1, the guide wire GW, which is disposed from the tip opening 164 to the side opening 165, pulls the tube wall toward the base end side A2. As a result, the collapsed conduit is pulled back to the base end side A2, returning to its original shape. The device configured to pull back the collapsed conduit is not limited to the guide wire GW, but may also be a device provided on the catheter 100.

FIG. 59 is a view showing an example of the biomanipulation process.

For example, as shown in FIG. 59, the surgeon uses the guide wire GW provided with a vibrator GWS at the tip. The surgeon inserts the guide wire GW into the conduit while vibrating the vibrator GWS to remove any collapse of the conduit. By removing the collapse, the guide wire GW can be inserted along the curve of the conduit of the bile duct B without collapsing it. If the guide wire GW does not have the vibrator GWS, the surgeon may vibrate the tip portion of the guide wire GW from the hand side.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, the fourth embodiment has been described in detail with reference to the drawings, but the specific configuration is not limited to this embodiment, and design changes that do not deviate from the spirit of the present disclosure are also included. In addition, the components shown in the above-mentioned embodiment and variant can be configured in any suitable combination.

### Fifth Embodiment

A cannulation method according to a fifth embodiment will be described with reference to FIG. 60 to FIG. 84. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S3E: Bile Duct Intubation Process>

In the case of the papilla PA, such as a long protrusion or intradiverticular papilla, the surgeon deforms the intubation device to fit the bile duct's conduit course, and inserts the device with the direction of advance aligned with the conduit course without readjusting the insertion shaft.

FIG. 60 is a view showing an example of the bile duct intubation process.

For example, as shown in FIG. 60, the surgeon injects, for example, a jelly-like liquid JE into the bile duct B. The liquid JE is injected deep along the bile duct B. The surgeon inserts the device using the liquid JE as a guide. The surgeon can insert the device along the conduit of the bile duct B without deforming the conduit. The liquid JE acts as a guide, allowing the device to be inserted regardless of the shape of the conduit. The liquid JE acts as a guide, making it difficult for the device to stack in the conduit. The liquid JE may be a drug that solidifies after a certain period of time after being injected into the bile duct B. The liquid JE may be, for example, liquid at room temperature and jelly-like at body temperature.

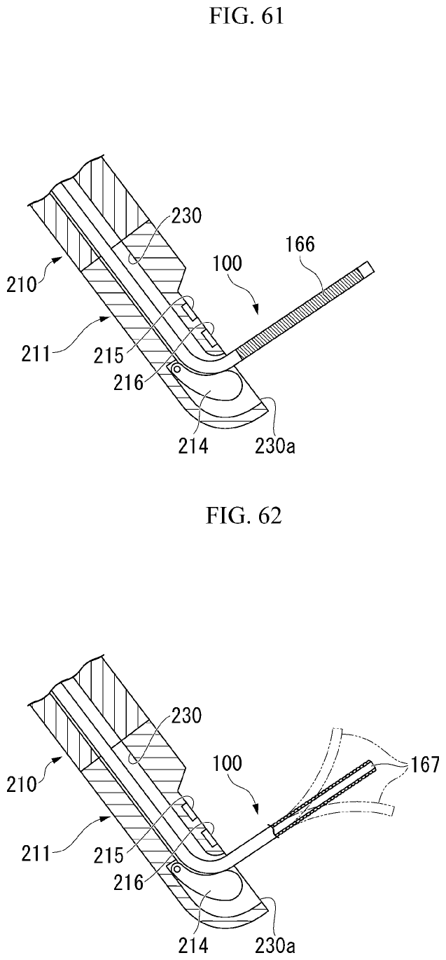

FIG. 61 is a view showing an example of the bile duct intubation process.

For example, as shown in FIG. 61, the surgeon uses the catheter 100 provided with a tip portion 166 formed by a coil spring. The surgeon can insert the device along the conduit of the bile duct B without deforming the conduit. The pushability of the tip portion 166 allows the device to be inserted regardless of the shape of the conduit. The pushability of the tip portion 166 makes it difficult for the device to get stuck in the conduit. Even when the insertion shaft of the catheter 100 and the axis of the conduit of the bile duct B are not aligned, the surgeon can easily insert the catheter 100.

FIG. 62 to FIG. 64 are views showing an example of the bile duct intubation process.

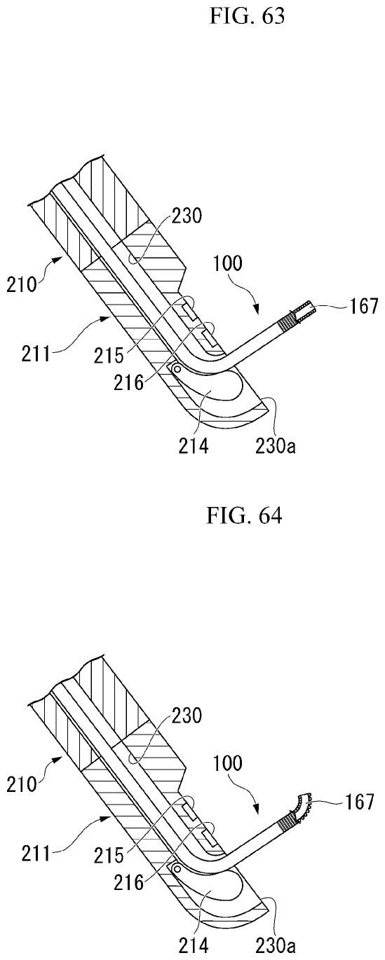

For example, as shown in FIG. 62, the surgeon uses the catheter 100 provided with a tip portion 167 formed in a bellows shape. Even when the insertion shaft of the catheter 100 and the axis of the conduit of the bile duct B are not aligned, the surgeon can easily insert the catheter 100. As shown in FIG. 63, the tip portion 167 can be contracted. As shown in FIG. 64, the tip portion 167 can bend in the contracted state. The tip portion 167 stretches and bends to stretch the conduit and allow intubation.

In the case of the papilla PA, such as a long protrusion or intradiverticular papilla, the surgeon uses an intubation device with a tip that generates a propulsive force, allowing the intubation device to self-propel within the canal and perform the intubation.

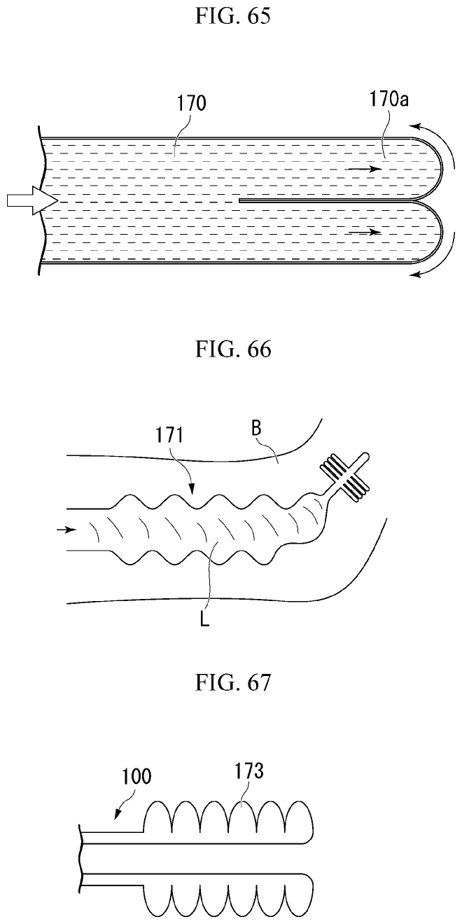

FIG. 65 is a view showing an example of the bile duct intubation process.

For example, as shown in FIG. 65, the surgeon uses a tube 170 with a tip portion 170a open. The surgeon inverts the tip portion 170a by injecting physiological salt solution into the tube 170. The tube 170 travels along a curved conduit. The physiological salt solution is also injected into the conduit. The tube 170 is less likely to get stuck in the conduit because it is not forced into the conduit. The surgeon inserts the guide wire GW into the bile duct B using the tube 170 inserted into the bile duct B. The surgeon may use the tube 170 to inject a contrast agent into the bile duct B.

FIG. 66 is a view showing an example of the bile duct intubation process.

For example, as shown in FIG. 66, the surgeon uses a tube 171 that is foldable and expandable. The surgeon can stretch the collapsed and contracted part of the tube 171 by injecting liquid L into the tube 171. The expanded tube 171 moves forward along the bile duct B. The tube 171 can move forward even if the bile duct B is bent significantly because the liquid L is injected into the bile duct B. The tube 171 is not pushed into the conduit, so it is less likely to get stuck in the conduit. The surgeon inserts the guide wire GW into the bile duct B using the tube 171 inserted into the bile duct B. The surgeon may inject a contrast agent into the bile duct B using the tube 171.

FIG. 67 is a view showing an example of the bile duct intubation process.

For example, as shown in FIG. 67, the surgeon uses the catheter 100 with a tip portion 173 that acts like a caterpillar. The tip portion 173 is extendable and bendable. The tip portion 173 can be inserted into a curved conduit without becoming stuck. The tip portion 173 can be configured with a balloon and inserted while expanding/contracting the balloon.

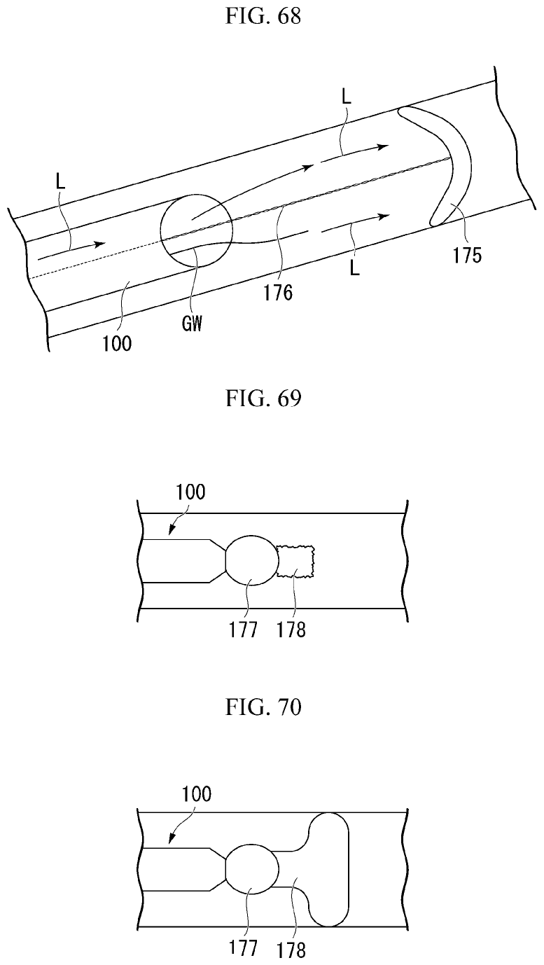

FIG. 68 is a view showing an example of the bile duct intubation process.

For example, as shown in FIG. 68, the surgeon uses the catheter 100 provided with an umbrella-shaped device 175. The umbrella-shaped device 175 is provided on the tip of the catheter 100 and is connected to the catheter 100 by a thread 176. The surgeon injects the liquid L into the catheter 100, causing the umbrella-shaped device 175 to move to the tip side Al by the liquid L. The umbrella-shaped device 175 can move forward along the bile duct B. The umbrella-shaped device 175 can move forward even if the bile duct B is greatly bent because the liquid L is injected into the bile duct B. The umbrella-shaped device 175 is inserted into the curved conduit without being stuck. The umbrella-shaped device 175 may be shaped like a windmill that is rotated by the flow of liquid L.

FIG. 69 to FIG. 71 are views showing an example of the bile duct intubation process.

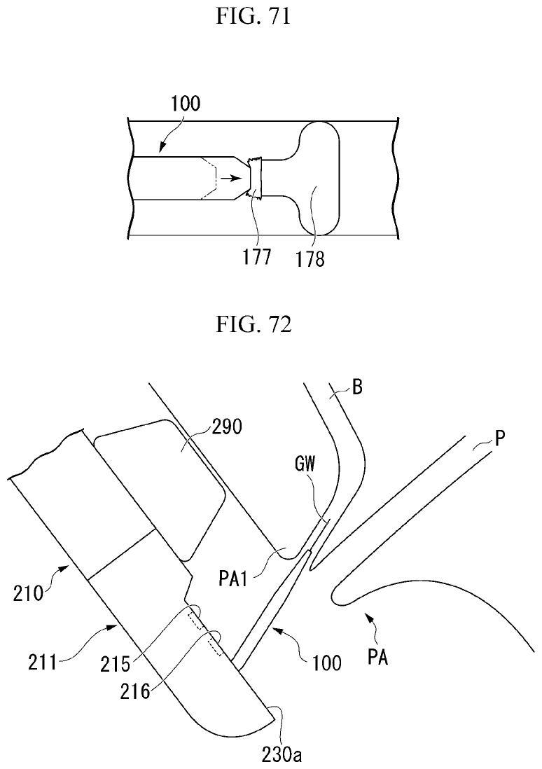

For example, as shown in FIG. 69 to FIG. 71, the surgeon uses the catheter 100 provided with a first balloon 177 and a second balloon 178. The first balloon 177 is provided on the tip of the catheter 100. The second balloon 178 is provided on the tip of the first balloon 177. As shown in FIG. 69, the surgeon inflates the first balloon 177. The inflated first balloon 177 extends along the longitudinal direction A. As shown in FIG. 70, the surgeon inflates the second balloon 178 until it contacts the tube wall. The inflated second balloon 178 extends in the radial direction perpendicular to the longitudinal direction A. Next, as shown in FIG. 71, the surgeon deflates the first balloon 177. Accordingly, the catheter 100 advances through the bile duct B. By repeating this process, the catheter 100 can advance through the bile duct B. The catheter 100 can be inserted into the curved conduit without becoming stuck.

In the case of the papilla PA such as a long protrusion, the surgeon uses the device to observe the lumen shape from outside the canal, check the bile duct course, and advance the intubation device in the direction of the bile duct course to perform the intubation.

FIG. 72 is a view showing an example of the bile duct intubation process.

For example, as shown in FIG. 72, the surgeon inserts the catheter 100 along the course of the bile duct B while observing the course of the bile duct B using an ultrasonic probe 290 attached to the endoscope 200. The surgeon can insert the catheter 100 along the conduit of the bile duct B without deforming the conduit.

In the case of the papilla PA such as a long protrusion, the surgeon inserts an imaging device into the canal, observes the lumen shape from inside the canal, checks the bile duct course, and advances the intubation device in the direction of the bile duct course to perform intubation.

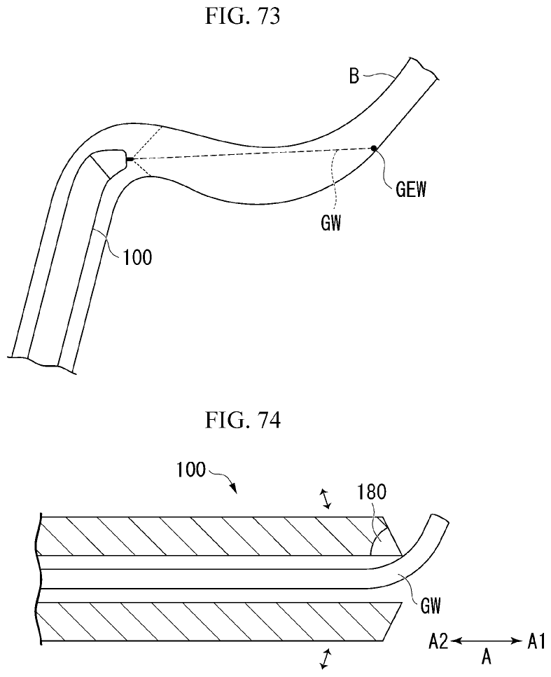

FIG. 73 is a view showing an example of the bile duct intubation process.

For example, as shown in FIG. 73, the surgeon uses the guide wire GW having an imaging function GEW and the catheter 100 having a steering function. The surgeon can insert the catheter 100 along the course of the bile duct B while observing the curved shape of the bile duct B from within the bile duct B. The surgeon can insert the catheter 100 along the conduit of the bile duct B without deforming the conduit.

FIG. 74 to FIG. 77 are views showing an example of the bile duct intubation process.

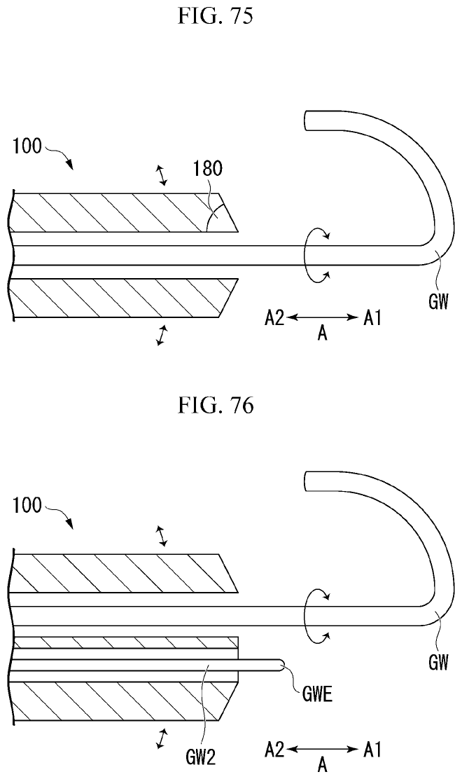

For example, as shown in FIG. 74, the surgeon uses the guide wire GW with a tendency to bend and the catheter 100 having an imaging function 180. The guide wire GW has a tendency to bend, and as shown in FIG. 75, it curves greatly when protruding from the tip of the catheter 100. The catheter 100 may have a bending function such as a sphincterotome. After inserting the catheter 100 into the opening portion PA1, the surgeon uses the imaging function 180 to check the course of the conduit. The surgeon rotates the catheter 100, aligns the tip of the guide wire GW with the direction of the conduit, and inserts the guide wire GW.

By using a standing platform 241 of the endoscope 200 in combination, the surgeon can further bend the direction of the catheter 100 or the guide wire GW.

As shown in FIG. 76, the surgeon may use a second guide wire GW2 having an imaging function GWE. If the catheter 100 has two lumens, the guide wire GW and the second guide wire GW2 can be inserted through separate lumens, respectively. When the second guide wire GW2 having the imaging function GWE is used, the catheter 100 does not need to have the imaging function 180.

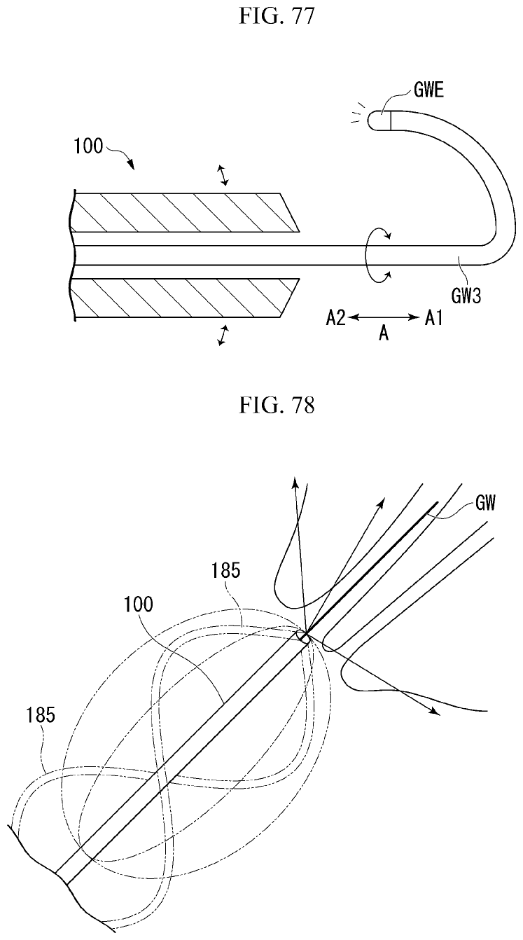

As shown in FIG. 77, the surgeon may use a third guide wire GW3, which has the imaging function GWE and tends to bend. In this case, the catheter 100 may not have the imaging function 180.

In the case of the papilla PA such as a long protrusion, the surgeon sets a rotation fulcrum of the device at the position of the papilla opening, eliminating the need for scope manipulation when adjusting the insertion shaft, and inserts the device in a direction of advance that matches the bile duct course.

FIG. 78 is a view showing an example of the bile duct intubation process.

For example, as shown in FIG. 78, the surgeon uses a direction change device 185 that can change the direction of the guide wire. The direction change device 185 can change the direction of the guide wire GW without moving the papilla PA. Since the papilla PA is not deformed, it is possible to prevent crushing of the conduit through which the guide wire GW is inserted.

In the case of the papilla PA with high mobility, the surgeon monitors the position and orientation of the papilla on the endoscopic image during intubation, and returns the papilla to its original position before intubation.

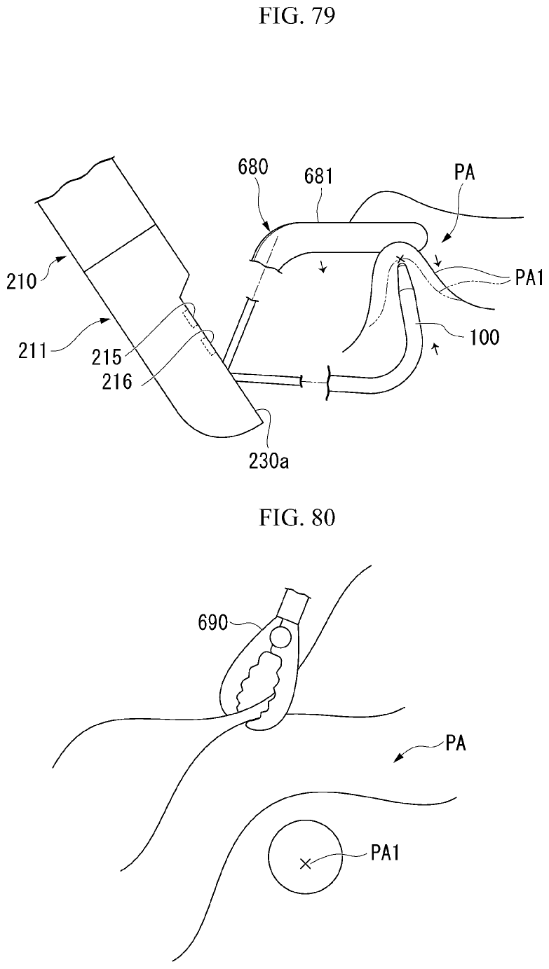

FIG. 79 is a view showing an example of the bile duct intubation process.

For example, as shown in FIG. 79, the surgeon uses the catheter 100 in combination with a spatula-shaped device 680. A tip portion 681 of the spatula-shaped device 680 is formed in a spatula shape. The surgeon presses the papilla PA with the tip portion 681 of the spatula-shaped device 680 to prevent the papilla PA from moving. In addition, if the papilla PA moves when inserting the catheter 100, the spatula-shaped device 680 is used to return the papilla to its original position and continue insertion.

For the papilla PA with high mobility, the surgeon uses a device to grasp the papilla or the vicinity thereof and fix the position of the papilla before intubation.

FIG. 80 is a view showing an example of the bile duct intubation process.

For example, as shown in FIG. 80, the surgeon uses a clip 690 or snare to hold down the tube wall and fix the papilla PA. Since the papilla PA does not move when inserting the guide wire GW or the like into the bile duct B, the surgeon can insert the guide wire GW or the like into the bile duct B reliably.

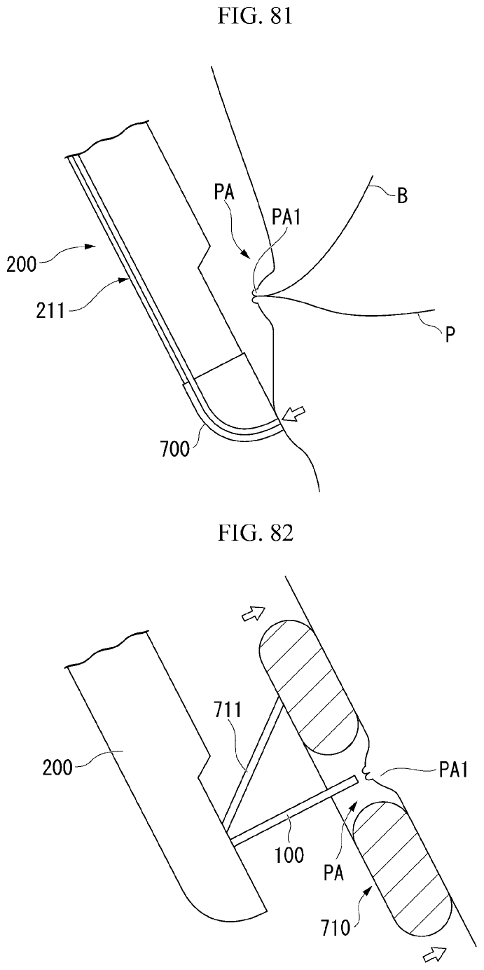

FIG. 81 is a view showing an example of the bile duct intubation process.

For example, as shown in FIG. 81, the surgeon uses a cap 700 that can be attached to the tip portion 211 of the endoscope 200. The cap 700 is provided with an external tube 701. The surgeon presses the cap 700 against the tube wall and uses the external tube 701 to suction the tube wall. Since the papilla PA is immobile, the surgeon can insert the guide wire GW or the like into the bile duct B.

FIG. 82 is a view showing an example of the bile duct intubation process.

For example, as shown in FIG. 82, the surgeon uses a balloon device 710. The balloon device 710 is formed in an annular (donut) shape. The balloon device 710 is provided with an air supply tube 711. The surgeon disposes the expanded balloon device 710 to surround the opening portion PA1 of the papilla PA. Since the papilla PA does not move, the surgeon can reliably insert the guide wire GW or the like into the bile duct B. The balloon device 710 may be formed in the shape of a cap, a snare, or a basket.

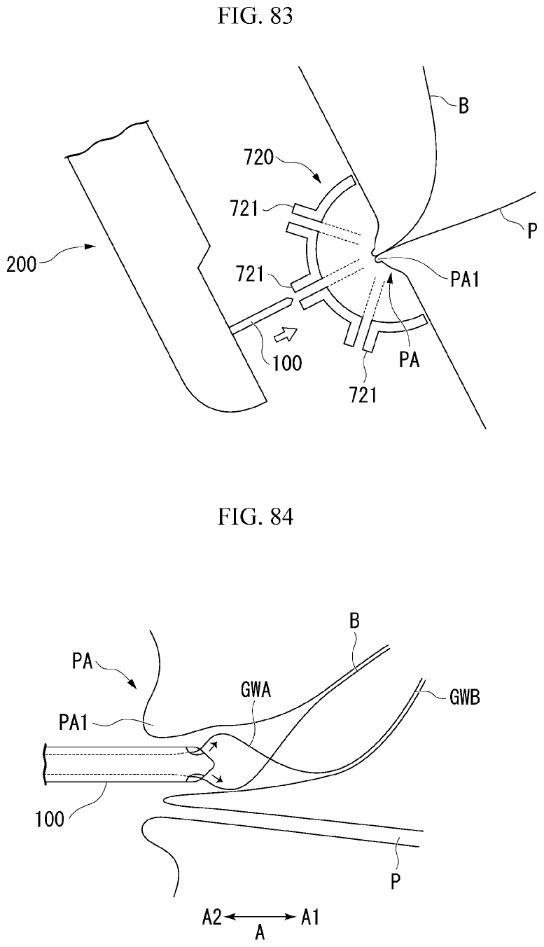

FIG. 83 is a view showing an example of the bile duct intubation process.

For example, as shown in FIG. 83, the surgeon uses a dome device 720. The dome device 720 is formed in a hemispherical (dome) shape. The dome device 720 has a plurality of through-holes 721 through which the catheter 100 and the guide wire GW can be inserted. The surgeon places the dome device 720 over the opening portion PA1 of the papilla PA. Since the papilla PA does not move, the surgeon can reliably insert the guide wire GW or the like into the bile duct B.

For the papilla PA with high mobility, the surgeon uses a device that can apply a force that offsets the force pushing against the papilla caused by the inserted device, preventing the papilla from moving during intubation.

FIG. 84 is a view showing an example of the bile duct intubation process.

For example, as shown in FIG. 84, the surgeon uses the catheter 100 having two guide wire lumens. A pair of guide wires GWA and GWB protrude from both sides of the central axis of the catheter 100 to the tip side A1. Since the pair of guide wires GWA and GWB protrude in opposite directions, the forces exerted by the pair of guide wires GWA and GWB on the tube wall are canceled out. As a result, since the papilla PA does not move, the surgeon can reliably insert the guide wire GW or the like into the bile duct B.

### No. 36

For the papilla PA with high mobility, during intubation, the surgeon monitors the force acting on the papilla and ensures that the force does not exceed a certain level, allowing the intubation to be performed without moving the papilla.

For example, a force sensor is attached near the tip of the catheter 100 or the guide wire GW to be inserted, and the signal from the sensor can be read through the endoscope 200 to monitor the force acting on the papilla PA during intubation. The surgeon performs the intubation while monitoring the amount of force acting on the papilla PA. If a force greater than the preset value is applied, the intubation operation is stopped and the intubation axis is readjusted so that a large force is not applied to the papilla. By stopping the pushing before a large force is applied to the papilla PA causing it to move, the surgeon can insert the guide wire GW or the like into the bile duct B without moving the papilla PA.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, the fifth embodiment has been described in detail with reference to the drawings, but the specific configuration is not limited to this embodiment, and design changes that do not deviate from the spirit of the present disclosure are also included.

### Sixth Embodiment

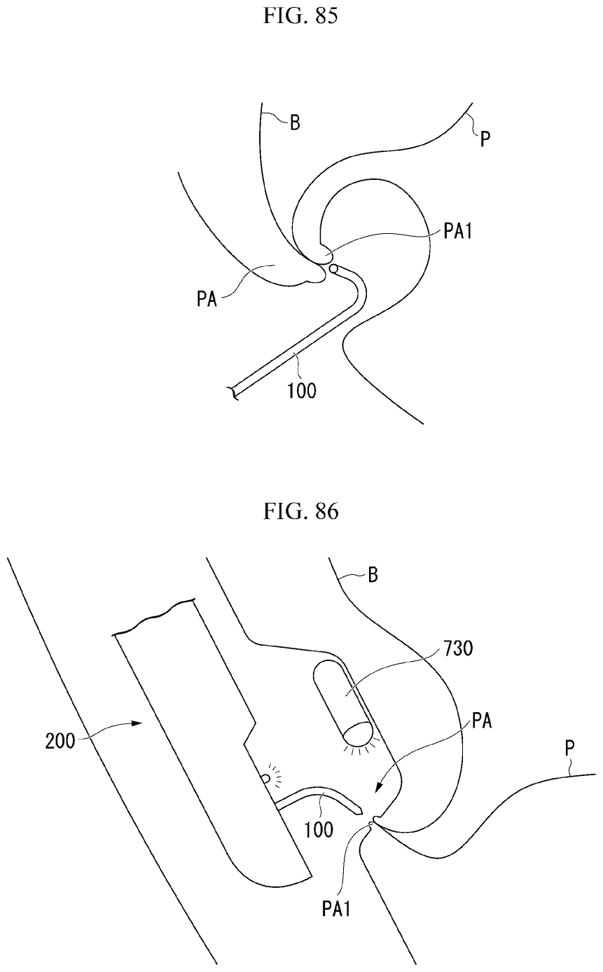

A cannulation method according to a sixth embodiment will be described with reference to FIG. 85 and FIG. 86. In the following description, for configurations that are common to those already described, the same reference signs will be used and duplicate descriptions will be omitted.

Actions and effects of the endoscope treatment system 300 according to the embodiment will be described. Specifically, a procedure for treating the bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

<step S3F: Observation Process>

In the case of the papilla PA, such as the intradiverticular papilla or the like, it may not be possible to check the position of the papilla PA. In this case, the surgeon inserts an imaging device into the treatment tool channel 230 of the endoscope 200 to observe the papilla PA.

FIG. 85 is a view showing an example of an observation process.

For example, as shown in FIG. 85, the surgeon uses the catheter 100 with a steering function. For example, as shown in FIG. 85, if the papilla PA is a paradiverticular papilla, the surgeon curves the catheter 100 with the steering function and places the tip portion into the diverticulum. The surgeon uses the guide wire GW, which has an imaging function, to observe the opening portion PA1 of the papilla PA. The surgeon allows observation of areas that cannot be observed with the endoscope 200. The guide wire GW may have a steering function.

In the case of the papilla PA, such as the intradiverticular papilla or the like, it may not be possible to check the position of the papilla PA. In this case, the surgeon uses the endoscope 200 to place an imaging device in a position where the papilla can be seen, and observes the papilla.

FIG. 86 is a view showing an example of the observation process.

For example, as shown in FIG. 86, the surgeon uses a capsule endoscope 730. The surgeon uses the endoscope 200 to place the capsule endoscope 730 in a position where the papilla PA can be observed. If the papilla PA is the paradiverticular papilla, the surgeon places the capsule endoscope 730 into the diverticulum. The surgeon allows observation of areas that cannot be observed with the endoscope 200.

### <Step S5: Contrast Process>

Step S5 is the same as in the first embodiment.

<Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinafter, the sixth embodiment has been described in detail with reference to the drawings, but the specific configuration is not limited to this embodiment, and design changes that do not deviate from the spirit of the present disclosure are also included. In addition, the components shown in the above-mentioned embodiment and variant can be configured in any suitable combination.

### Seventh Embodiment

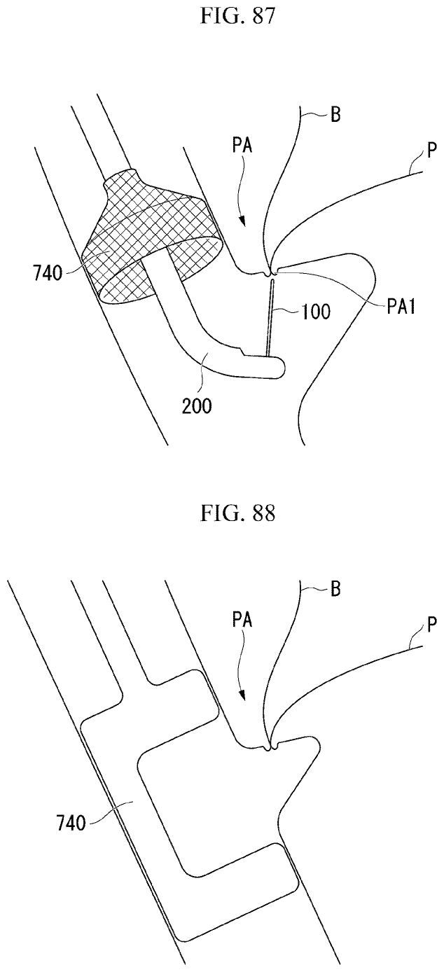

A cannulation method according to a seventh embodiment will be described with reference to FIG. 87 and FIG. 88. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

<step S3G: Observation Process>

In the case of the papilla PA, such as the intradiverticular papilla or the like, it may not be possible to check the position of the papilla PA. In this case, the surgeon uses the endoscope 200 to steadily observe the papilla PA.

FIG. 87 and FIG. 88 are views showing an example of the observation process.

For example, as shown in FIG. 87, the surgeon uses a stent-like device 740. The surgeon places the stent-like device 740 in the duodenum, anterior to the diverticulum, and supports the endoscope 200 with the reduced diameter portion of the stent-like device 740. Since the endoscope 200 is supported by the stent-like device 740, the surgeon can easily change the direction or posture of the endoscope 200 according to his or her intention. The surgeon can maintain a constant distance between the papilla and the endoscope 200. The surgeon makes it easier to observe areas that cannot be observed with the operated endoscope 200 without the stent-like device 740. The stent-like device 740 may be U-shaped as shown in FIG. 88.

In the case of the papilla PA, such as the intradiverticular papilla or the like, it may not be possible to check the position of the papilla PA. In this case, the surgeon places a mirror at the bottom of the diverticulum and observes the reflected image of the papilla through a scope.

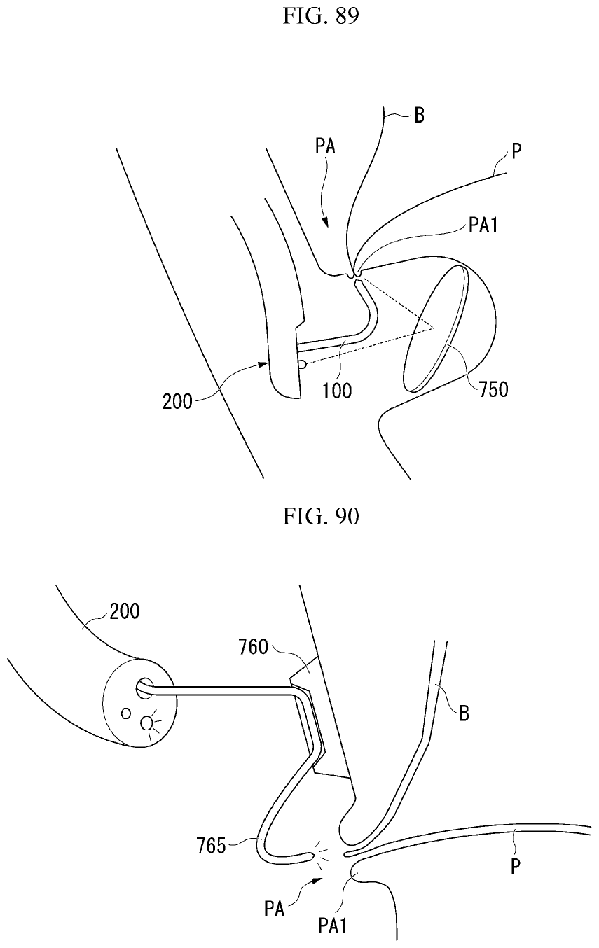

FIG. 89 is a view showing an example of the observation process.

For example, as shown in FIG. 89, the surgeon uses a mirror device 750. The surgeon places the mirror device 750 at the bottom of the diverticulum and observes the papilla PA using the reflected image of the mirror device 750. The image captured from the reflected image of the mirror device 750 may be converted using image processing techniques, etc., to a normally observed endoscope image. The surgeon allows observation of areas that cannot be observed with the endoscope 200.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the seventh embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Eighth Embodiment

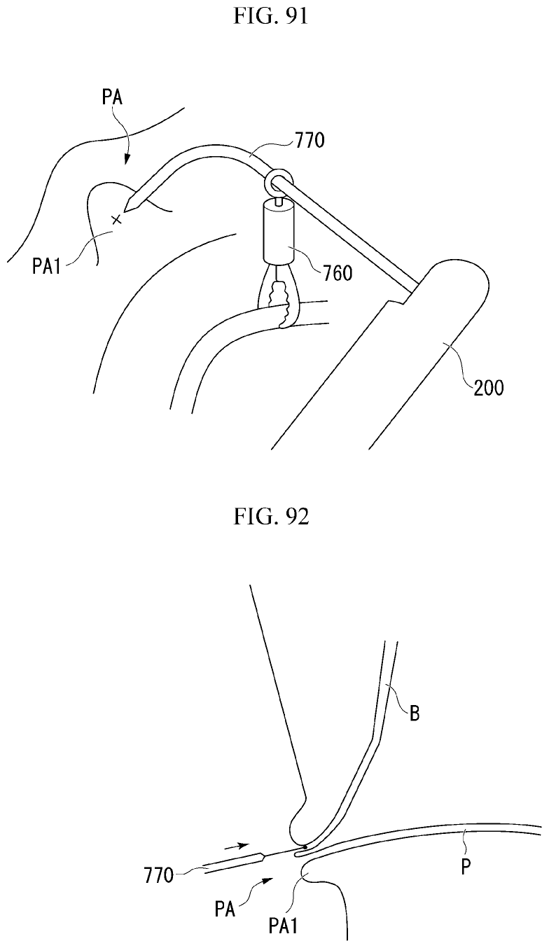

A cannulation method according to an eighth embodiment will be described with reference to FIG. 90 and FIG. 91. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S3H: Observation Process>

In the case of the papilla PA, such as the intradiverticular papilla or the like, it may not be possible to check the position of the papilla PA. In this case, the surgeon places a device on the duodenum tube wall near the papilla PA as a relay station for the procedure, and directs an imaging device from the relay station to observe the papilla PA.

FIG. 90 and FIG. 91 are views showing an example of the observation process.

For example, as shown in FIG. 90, the surgeon uses a relay device 760. The surgeon places the relay device 760 near the papilla PA. The surgeon places an imaging device 765 with a steering function near the papilla PA through the relay device 760 to observe the papilla PA. If the papilla PA is a paradiverticular papilla, the surgeon places the relay device 760 near the entrance to the diverticulum. The surgeon can perform observations using the same procedure regardless of whether the diverticulum is present or not. The surgeon allows observation of areas that cannot be observed with the endoscope 200. The relay device 760 may include a clip as shown in FIG. 91.

In addition, even without creating a point like the relay device 760, the papilla PA can be observed using a device that moves along the inner wall surface of the duodenum.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the eighth embodiment has been described in detail with reference to the drawings, the specific configuration is not limited to this embodiment, and design changes within the scope that do not deviate from the spirit of the present disclosure are also included. In addition, the components shown in the above-mentioned embodiment and variant can be configured in any suitable combination.

### Ninth Embodiment

A cannulation method according to a ninth embodiment will be described with reference to FIG. 92 to FIG. 95. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S3H: Insertion Process>

In the papilla PA such as the septal type or the like, the septum S is located between the bile duct B and the pancreatic duct P within the opening portion PA1, and the opening of the bile duct B is narrower than the opening of the pancreatic duct P, making it difficult to insert the device into the bile duct B. In this case, the surgeon inserts the device directly above the opening portion PA1 of the papilla PA, gripping only the tip portion of the device, and then sharpens the insertion angle of the device to perform intubation.

FIG. 92 to FIG. 94 are views showing an example of an insertion process.

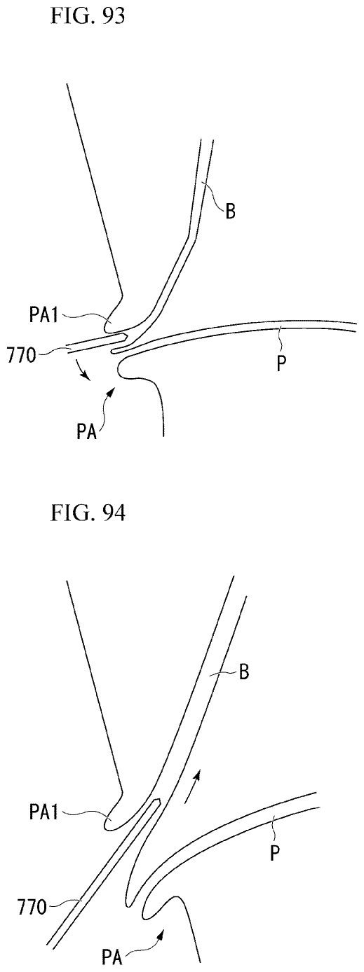

For example, as shown in FIG. 92 to FIG. 94, the surgeon uses a flexible device 770. The flexible device 770 includes the catheter 100, the guide wire GW, or the like. As shown in FIG. 92, the surgeon slightly inserts the flexible device 770 perpendicular to the papilla PA. Next, as shown in FIG. 93, the surgeon points the flexible device 770 in a direction that looks up at the papilla PA. Next, as shown in FIG. 94, the surgeon inserts the flexible device 770. When inserting the flexible device 770, the area of the opening portion PA1 becomes larger than when inserting it from the beginning in the looking-up direction, making it easier for the surgeon to insert the flexible device 770.

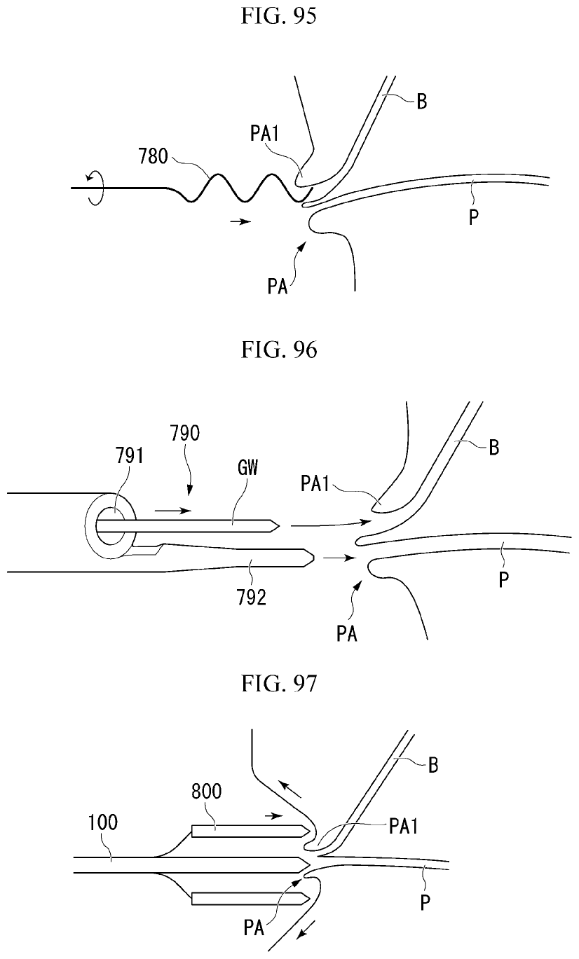

FIG. 95 is a view showing an example of the insertion process.

For example, as shown in FIG. 95, the surgeon uses a spiral device 780 with a spiral tendency to bend. The spiral device 780 includes the catheter 100, the guide wire GW, or the like. The surgeon rotates and thrusts the spiral device 780 to approach the opening portion PA1 of the papilla PA. The spiral direction of the spiral device 780 relative to the opening portion PA1 of the papilla PA is always at a steep angle. The device 780, which has a spiral tendency to bend, can be projected from the endoscope 200 at a gentle angle, making it easier for the surgeon to perform the insertion operation.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the ninth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Tenth Embodiment

A cannulation method according to a tenth embodiment will be described with reference to FIG. 96. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S3H: Pancreatic Duct Obstruction Process>

In the papilla PA such as the septal type or the like, the septum S is located between the bile duct B and the pancreatic duct P within the opening portion PA1, and the opening of the bile duct B is narrower than the opening of the pancreatic duct P, making it difficult to insert the catheter into the bile duct B. In this case, the surgeon closes the opening of the pancreatic duct P, eliminating misinsertion into the pancreatic duct P.

FIG. 96 is a view showing an example of a pancreatic duct obstruction process.

For example, as shown in FIG. 96, the surgeon uses a bifurcated device 790. The bifurcated device 790 has a lumen 791 through which the guide wire GW is inserted, and a rod 792 protruding toward the tip side A1. The surgeon inserts the rod 792 into the pancreatic duct P to block the pancreatic duct P. Next, the surgeon inserts the guide wire GW into the bile duct B.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the tenth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Eleventh Embodiment

A cannulation method according to an eleventh embodiment will be described with reference to FIG. 97 to FIG. 101. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S3H: Insertion Process>

The papilla PA, such as a small papilla or the like, is difficult to see and difficult to insert into the opening portion PA1. In this case, the surgeon may use a device that makes it easier to insert into the bile duct B, or may use a device to make it easier to insert into the bile duct.

FIG. 97 is a view showing an example of the insertion process.

For example, as shown in FIG. 97, the surgeon uses a cap device 800. The surgeon places the cap device 800 over the papilla PA and pushes it therein. The papilla PA is pushed in, widening the opening portion PA1, making it easier for the surgeon to insert the guide wire GW or the like.

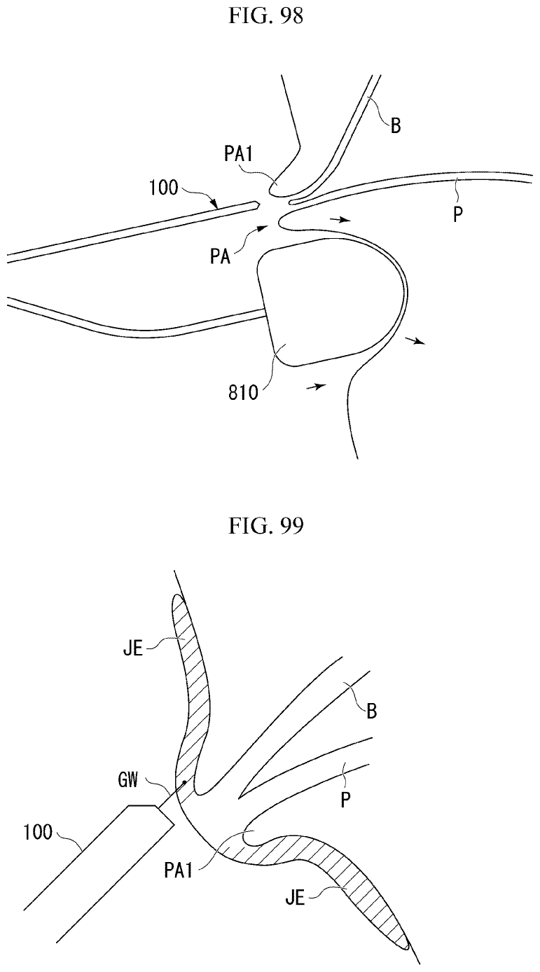

FIG. 98 is a view showing an example of the insertion process.

For example, as shown in FIG. 98, the surgeon uses a balloon device 810. The surgeon expands the balloon device 810 to push into the septum. The papilla PA is pushed in, widening the opening portion PA1, making it easier for the surgeon to insert the guide wire GW or the like.

FIG. 99 is a view showing an example of the insertion process.

For example, as shown in FIG. 99, the surgeon applies the highly viscous liquid JE, which is like a jelly, to the area around the papilla PA. The surgeon places the guide wire GW or other delivery device near the papilla PA, moves it over the surface of the papilla PA, and searches for the opening portion (PA1) through which to insert it. The guide wire GW moved to the opening portion PA1 is smoothly inserted into the bile duct B. The surgeon can insert the guide wire GW into the bile duct B even when the location of the opening portion PA1 cannot be clearly determined.

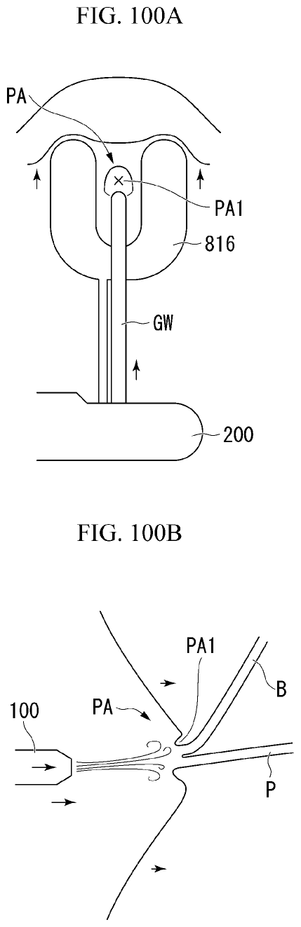

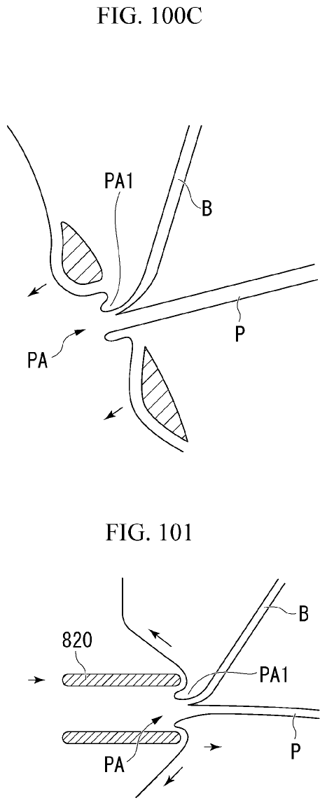

FIGS. 100A and 100B are views showing an example of the insertion process.

For example, as shown in FIG. 100A, the surgeon pushes the periphery of the papilla PA with a push device 816, causing a depression in the periphery of the papilla PA. Alternatively, as shown in FIG. 100B, the surgeon blows air into the periphery of the papilla PA, temporarily creating a depression in the periphery of the papilla PA. The surgeon can precisely target the bile duct B and insert the guide wire GW or the like.

Alternatively, as shown in FIG. 100C, the surgeon uses the local injection device 500 to perform a local injection near the papilla PA, enlarging the surroundings and thereby enlarging the papilla PA. In addition, the surgeon then performs further local injection using the local injection device 500 to enlarge the surroundings and depress the papilla PA. Accordingly, the papilla PA becomes easier to see and easier to align.

FIG. 101 is a view showing an example of the insertion process.

For example, as shown in FIG. 101, the surgeon places a tubular-shaped cap 820 over the papilla PA and pushes the papilla PA outward in the circumferential direction to expand it. Alternatively, the surgeon places a balloon over the papilla PA and pushes it outward in the circumferential direction to expand it. The tube wall is stretched and the papilla is also stretched laterally. Accordingly, the distance between the opening portion of the bile duct B and the opening portion of the pancreatic duct P can be increased, making it easier for the surgeon to insert the guide wire GW or the like, aiming at the bile duct B.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the eleventh embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Twelfth Embodiment

A cannulation method according to a twelfth embodiment will be described. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

<Step S2: endoscope positioning process>

Step S2 is the same as in the first embodiment.

<Step S3H: Insertion Process>

In the case of the papilla PA, such as a septal type or small papilla, the surgeon brings the imaging device close to the opening portion PA1 of the bile duct B to observe the opening portion PA1 of the papilla PA in detail and ensure accurate intubation of the device into the bile duct B.

For example, the surgeon uses the guide wire GW, which has the imaging function GWE, to protrude the guide wire GW into the papilla PA while observing the papilla PA. The surgeon can check the forward direction of the guide wire GW, so that the guide wire GW can be inserted reliably into the opening portion PA1 of the bile duct B. This prevents the guide wire GW from being accidentally inserted into the pancreatic duct P. The surgeon may use the imaging function of other devices, such as the catheter 100 or the like.

In addition, the surgeon uses an insertion device with a shape that makes it easy to recognize the position and orientation of the tip portion. The insertion device includes the catheter 100, the guide wire GW, or the like. A shape that makes it easy to recognize the position and orientation of the tip portion is, for example, a pyramidal shape that has ridges. The surgeon makes it easy to check the orientation, position, or posture of the insertion device, and to perform alignment and correction. For this reason, the surgeon can accurately target the bile duct B and insert the insertion device.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the twelfth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Thirteenth Embodiment

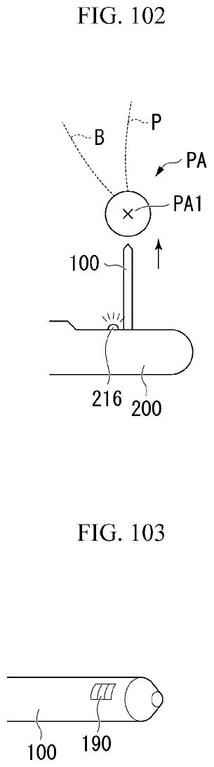

A cannulation method according to a thirteenth embodiment will be described with reference to FIG. 102. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

### <Step S3H: Insertion Process>

When performing cannulation, if it detects that the insertion device is in a position that makes it easy to insert into the pancreatic duct P, the surgeon is notified and the intubation is stopped.

FIG. 102 is a view showing an example of the insertion process.

For example, as shown in FIG. 102, the surgeon uses a determination mechanism to determine whether the catheter 100 is pointing toward the bile duct B. The determination mechanism is, for example, a mechanism that calculates the orientation of the catheter 100 from the image captured by the optical observation part 216 of the endoscope 200. This prevents the guide wire GW from being accidentally inserted into the pancreatic duct P. The determination mechanism may be included in an endoscopic treatment tool such as the catheter 100.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the thirteenth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Fourteenth Embodiment

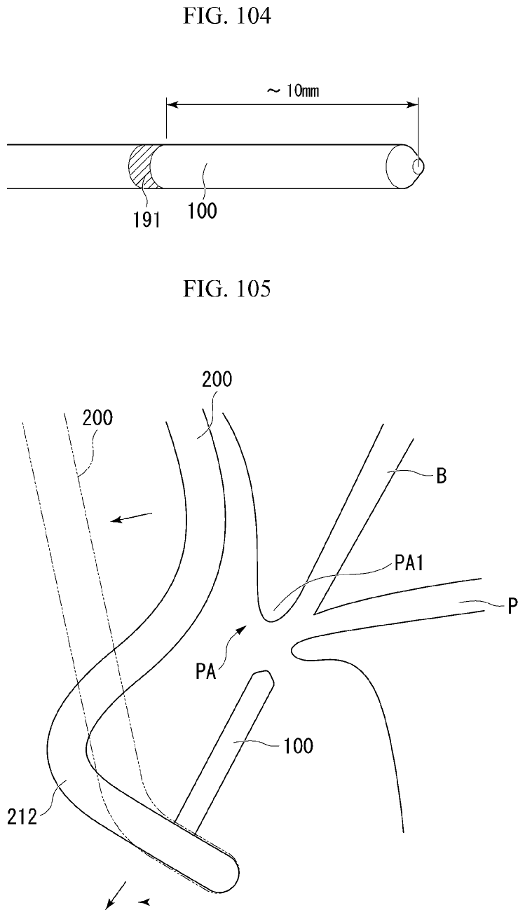

A cannulation method according to a fourteenth embodiment will be described with reference to FIG. 103 and FIG. 104. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

<step S3H: Insertion Process>

When performing cannulation, it is checked that the insertion device has entered the bile duct B before continuing with intubation.

FIG. 103 is a view showing an example of the insertion process.

For example, as shown in FIG. 103, the surgeon uses the catheter 100 equipped with a bile sensor 190 at the tip portion. The bile sensor 190 is a sensor configured to detect the bile. Based on the detection result of the bile sensor 190, the surgeon checks that the tip portion of the catheter 100 is positioned at the opening portion PA1 of the bile duct B, and then inserts the catheter 100 into the bile duct B. The surgeon can reliably insert the catheter 100 into the bile duct B. This prevents the catheter 100 from being accidentally inserted into the pancreatic duct P.

FIG. 104 is a view showing an example of the insertion process.

For example, as shown in FIG. 104, the surgeon uses the catheter 100 equipped with a marker 191 at the tip portion. The marker 191 may be a marker formed in a color that is easy to observe on an endoscope image, such as green or black, or may be a marker that can be detected by X-ray. The marker 191 is located, for example, 10 mm from the tip of the catheter 100 toward the base end side A2. The surgeon can adjust the insertion depth of the catheter 100 by checking the position of the marker 191. This prevents the catheter 100 from entering deeper into the pancreatic duct P than expected. The guide wire GW may have the marker 191.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the fourteenth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Fifteenth Embodiment

A cannulation method according to the fifteenth embodiment will be described. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

<step S3H: Insertion Process>

When performing cannulation, the device detects that the insertion device has entered the pancreatic duct P and notifies the user to stop the intubation.

For example, the surgeon uses the catheter 100 with the imaging function 180 to detect that the catheter 100 has been incorrectly inserted into the pancreatic duct P. The surgeon stops the intubation procedure, preventing the catheter 100 or the guide wire GW from being inserted too deep into the pancreatic duct P by mistake.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the fifteenth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Sixteenth Embodiment

A cannulation method according to a sixteenth embodiment will be described with reference to FIG. 105. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

### <Step S1: Endoscope Insertion Process>

Step S1 is the same as in the first embodiment.

### <Step S2: Endoscope Positioning Process>

Step S2 is the same as in the first embodiment.

<step S3H: Insertion Process>

When positioning the endoscope 200 for the papilla PA, such as an intradiverticular papilla, if the bending function of the curved section 212 has been used to its maximum extent, the surgeon uses a system that automatically controls the posture of the endoscope 200 to reduce the curved shape of the endoscope 200 without changing the insertion direction of the insertion device, so that the range of movement for endoscopic manipulation can always be maintained.

FIG. 105 is a view showing an example of the insertion process.

For example, as shown in FIG. 105, by loosening the curved section 212 of the endoscope 200 while keeping the catheter 100 pointed toward the bile duct B, the surgeon can use a curving mechanism of the curved section 212 to align the catheter 100 when it becomes necessary to align the endoscope 200 in the future.

In addition, the control range of the endoscope 200 is kept limited at all times, allowing for a greater bending range of the endoscope when needed.

For example, it is possible to make it easier to align the catheter 100 by partially limiting the curvature range of the curved section 212 in advance and then removing this limitation when necessary.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the sixteenth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

### Seventeenth Embodiment

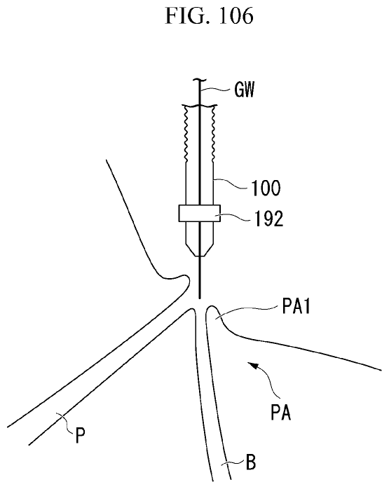

A cannulation method according to a seventeenth embodiment will be described with reference to FIG. 106. In the following description, for configurations that are common to those already described, the same reference signs will be used and duplicate descriptions will be omitted.

Actions and effects of the endoscope treatment system 300 according to the embodiment will be described. Specifically, a procedure for treating bile duct B using endoscopic retrograde cholangiopancreatography (ERCP) will be described.

<step S1: Endoscope Insertion Process>

Step S1 is the same as in the first embodiment.

### <Step S2: Endoscope Positioning Process>

Step S2 is the same as in the first embodiment.

### <Step S3H: Insertion Process>

The papilla PA, such as a small papilla or the like, is difficult to see and difficult to insert into the opening portion PA1. In this case, the surgeon guides the intubation device into the bile duct opening.

FIG. 106 is a view showing an example of the insertion process.

For example, as shown in FIG. 106, the surgeon uses the catheter 100 equipped with a weight 192 at the tip portion. The surgeon adjusts the patient's posture so that the course of the bile duct B faces vertically downward. The surgeon uses gravity acting on the weight 192 to move the weight 192 toward the opening portion PA1. The tip portion with the weight 192 attached enters the bile duct B due to its own weight. Since the course of the canals other than bile duct B is not vertically downward, the tip portion with weight 192 attached does not enter those canals. Even if the surgeon cannot grasp the opening of the bile duct B, he or she can intubate the tip portion of the catheter 100. This prevents the catheter 100 from being accidentally inserted into the pancreatic duct P.

A similar mechanism can also be used to induce magnetic fields.

### <Step S5: Contrast Process>

Step S5 is the same as in the first embodiment.

### <Step S6: Guide Wire Indwelling Process>

Step S6 is the same as in the first embodiment.

According to the insertion method into the bile duct B according to the embodiment, the guide wire GW is easy to be inserted into the bile duct. In addition, the guide wire GW inserted into the bile duct makes it easy to insert a medical device such as a stent delivery device or the like into the bile duct.

Hereinabove, although the seventeenth embodiment has been described in detail with reference to the drawings, the specific configuration is not limited to this embodiment, and design changes that do not deviate from the spirit of the present disclosure are also included. In addition, the components shown in the above-mentioned embodiment and variant can be configured in any suitable combination.

In the above-mentioned embodiment, the guide wire GW or the like is inserted into the bile duct B. However, the cannulation method in the above-mentioned embodiment can also be used to insert the guide wire GW or the like into the pancreatic duct P.

What is claimed is:

1. A cannulation method comprising:
inserting an endoscope close to the papilla;
deforming the papilla or bile pancreatic duct; and
inserting an insertion device into the bile pancreatic duct.

2. The cannulation method according to claim 1, further comprising:
manipulating the papilla to move an opening portion of the bile pancreatic duct to a position seen from the endoscope; and
inserting the insertion device into the opening portion.

3. The cannulation method according to claim 2, wherein,
by grasping and pulling a tube wall, the opening portion of the bile pancreatic duct is moved to a position seen from the endoscope.

4. The cannulation method according to claim 2, wherein,
by injecting the papilla locally, the opening portion of the bile pancreatic duct is moved to a position visible through the endoscope.

5. The cannulation method according to claim 2, wherein,
by expanding a balloon, the papilla is deformed, and the opening portion of the bile pancreatic duct is moved to a position visible through the endoscope.

6. The cannulation method according to claim 2, wherein,
by expanding a stent, the papilla is deformed, and the opening portion of the bile pancreatic duct is moved to a position visible through the endoscope.

7. The cannulation method according to claim 1, further comprising:
immobilizing the papilla and inserting the insertion device into the opening portion of the bile pancreatic duct.

8. The cannulation method according to claim 7, wherein
the papilla is made less likely to move by spraying the opening portion with cold water, salt, or a coating agent.

9. The cannulation method according to claim 1, further comprising:
directing the insertion device toward the opening portion of the bile pancreatic duct while moving an obstacle that hinders insertion of the insertion device into the bile pancreatic duct; and
inserting the insertion device into the opening portion.

10. The cannulation method according to claim 9, wherein
the obstacle is moved by removing at least a portion of the obstacle.

11. The cannulation method according to claim 1, further comprising:
widening the opening portion of the bile pancreatic duct; and
inserting the insertion device into the widened opening portion.

12. The cannulation method according to claim 11, wherein
one canal of the bile pancreatic duct is suctioned to widen the opening portion of the other canal of the bile pancreatic duct.

13. The cannulation method according to claim 11, wherein
the papilla is pushed or clamped to widen the opening portion of the bile pancreatic duct.

14. The cannulation method according to claim 1, further comprising:
acting on the living organism to relax the angle of the bile duct course relative to the duodenum course, and
inserting the insertion device into the opening portion of the bile pancreatic duct.

15. The cannulation method according to claim 14, wherein
a periphery of the papilla is pulled to relax the angle of the bile duct course relative to the duodenum course.

16. The cannulation method according to claim 1, further comprising:
pulling the periphery of the papilla to deform the bile pancreatic duct into a straight line; and
inserting the insertion device into the opening portion of the bile pancreatic duct.

17. The cannulation method according to claim 16, wherein
a tensioning device with a hook at the tip is hooked inside the papilla, and the tensioning device is pulled to deform the bile pancreatic duct into a straight line.

18. The cannulation method according to claim 16, wherein
an area near the papilla is grasped with a grasping device, and the grasping device is pulled to deform the bile pancreatic duct into a straight line.

19. The cannulation method according to claim 1, further comprising:
inserting the insertion device while straightening the curved portion of the bile pancreatic duct each time the insertion device passes therethrough.

20. The cannulation method according to claim 1, further comprising:
pulling back the tube wall to insert the insertion device while pulling the bile pancreatic duct.
